(19) United States
(12) Patent Application Publication
(10) Pub. No.: US 2025/0134803 A1
(43) Pub. Date: May 1, 2025

(54) TOPICAL DIM COMPOSITIONS AND USES THEREOF

(71) Applicant: SKINTECH LIFE SCIENCE LIMITED, Charlotte, NC (US)

(72) Inventors: David Alpert, London (GB); Gabriell Prinsloo, London (GB)

(21) Appl. No.: 18/935,357

(22) Filed: Nov. 1, 2024

Related U.S. Application Data
(60) Provisional application No. 63/546,888, filed on Nov. 1, 2023.

(51) Int. Cl. A61K 9/00; A61K 9/06; A61K 9/107; A61K 31/404; A61K 45/06; A61K 47/10; A61K 47/12; A61K 47/14; A61K 47/18; A61K 47/22; A61K 47/24; A61K 47/32; A61K 47/36; A61K 47/44

(52) U.S. Cl. CPC A61K 9/0014; A61K 9/06; A61K 9/107; A61K 31/404; A61K 45/06; A61K 47/10; A61K 47/12; A61K 47/14; A61K 47/186; A61K 47/22; A61K 47/24; A61K 47/32; A61K 47/36; A61K 47/44; A61K 2121/00

(57) ABSTRACT

Described herein are methods for treating one or more skin conditions by administering, via the topical route, a composition comprising substituted or unsubstituted diindolylmethane. In particular, methods are provided for improving the dermal bioavailability and pharmacokinetic parameters of substituted or unsubstituted diindolylmethane following a topical administration, relative to an oral administration or a sublingual or buccal administration.

### CROSS-REFERENCE

This application claims the benefit of U.S. Provisional Application No. 63/546,888 filed on Nov. 1, 2023, which is incorporated by reference herein in its entirety.

### BACKGROUND OF THE DISCLOSURE

Skin diseases and conditions, such as those associated inflammation and/or irritation, affect a large population of the world. Acne is a skin condition characterized by areas of blackheads, whiteheads, pimples, greasy skin, and possibly scarring. Atopic dermatitis, often referred to as eczema, is a chronic (long-lasting) disease that causes inflammation, redness, and irritation of the skin. It is a common condition that usually begins in childhood; however, anyone can get the disease at any age. Rosacea is a chronic skin condition characterized by facial redness, small and superficial dilated blood vessels on facial skin, papules, pustules, and swelling. Skin aging also affects a large population of the world.

### SUMMARY OF THE DISCLOSURE

One aspect of the present disclosure provides a method of treating a skin condition or skin aging in a subject in need thereof comprising administering to the subject a topical composition comprising. The topical composition comprises emulsified substituted or unsubstituted diindolylmethane and a pharmaceutically acceptable carrier, and wherein the composition provides therapeutically effective amount of the substituted or unsubstituted diindolylmethane in the subject's skin affected by the skin disease or condition.

One aspect of the present disclosure provides a method of treating a skin condition or skin aging in a subject in need thereof comprising administering to the subject a topical composition comprising a substituted or unsubstituted diindolylmethane. The composition comprises from about 0.1 wt % to about 10 wt % of substituted or unsubstituted diindolylmethane and a pharmaceutically acceptable carrier, and the composition provides therapeutically effective amount of the substituted or unsubstituted diindolylmethane in the subject's skin affected by the skin disease or condition.

One aspect of the present disclosure provides a method of treating a skin condition or skin aging in a subject in need thereof comprising administering to the subject a topical composition comprising a substituted or unsubstituted diindolylmethane. The composition comprises from about 0.1 wt % to about 10 wt % of emulsified substituted or unsubstituted diindolylmethane and a pharmaceutically acceptable carrier, and the composition provides therapeutically effective amount of the substituted or unsubstituted diindolylmethane in the subject's skin affected by the skin disease or condition.

Provided herein in one embodiment is a method of treating acne in a subject in need thereof by topically administering the topical composition disclosed herein. Provided herein in one embodiment is a method of treating atopic dermatitis in a subject in need thereof by topically administering the topical composition disclosed herein. Provided herein in one embodiment is a method of treating rosacea in a subject in need thereof by topically administering the topical composition disclosed herein. Provided herein in one embodiment is a method of treating skin aging in a subject in need thereof by topically administering the topical composition disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates the structure of skin, particularly showing the epidermis, dermis and hypodermis layers;

FIG. 2 schematically illustrate the microdialysis and tape striping technique;

FIG. 3 is the imaging result of a patient using a non-limiting embodiment of the disclosed DIM topical formulation, showing improved in skin condition;

FIG. 4 is the imaging result of a patient using a non-limiting embodiment of the disclosed DIM topical formulation, showing improved in skin condition;

FIG. 5 is the imaging result of a patient using a non-limiting embodiment of the disclosed DIM topical formulation, showing improved in skin condition;

FIG. 6 is the imaging result of a patient using a non-limiting embodiment of the disclosed DIM topical formulation, showing improved in skin condition;

FIG. 7 is the imaging result of a patient using a non-limiting embodiment of the disclosed DIM topical formulation, showing improved in skin condition;

FIG. 8 is the imaging result of a patient using a non-limiting embodiment of the disclosed DIM topical formulation, showing improved in skin condition;

FIG. 9 is the imaging result of a patient using a non-limiting embodiment of the disclosed DIM topical formulation, showing improved in skin condition;

FIG. 10 is the imaging result of a patient using a non-limiting embodiment of the disclosed DIM topical formulation, showing improved in skin condition;

FIG. 11 is the imaging result of a patient using a non-limiting embodiment of the disclosed DIM topical formulation, showing improved in skin condition;

FIG. 12 is the imaging result of a patient using a non-limiting embodiment of the disclosed DIM topical formulation, showing improved in skin condition;

FIG. 13 is the imaging result of a patient using a non-limiting embodiment of the disclosed DIM topical formulation, showing improved in skin condition;

FIG. 14 is the imaging result of a patient using a non-limiting embodiment of the disclosed DIM topical formulation, showing improved in skin condition;

FIG. 15 is the imaging result of a patient using a non-limiting embodiment of the disclosed DIM topical formulation, showing improved in skin condition;

FIG. 16 is the imaging result of a patient using a non-limiting embodiment of the disclosed DIM topical formulation, showing improved in skin condition; and

FIG. 17 is the imaging result of a patient using a non-limiting embodiment of the disclosed DIM topical formulation, showing improved in skin condition.

### DETAILED DESCRIPTION OF THE DISCLOSURE

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed. It is intended that the following claims define the scope of the present disclosure and that methods and structures within the scope of these claims and their equivalents be covered thereby.

Unless defined otherwise, all technical and scientific terms used herein have the same meaning as commonly understood by one of skill in the art to which the present disclosure described herein belong. All publications, patents, and patent applications mentioned in this specification are hereby incorporated by reference to the same extent as if each individual publication, patent, or patent application was specifically and individually indicated to be incorporated by reference.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

### Certain Definitions

Unless defined otherwise, all technical and scientific terms used herein have the same meaning as is commonly understood by one of skill in the art to which the claimed subject matter belongs. In the event that there is a plurality of definitions for terms herein, those in this section prevail.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting.

As used herein, the term “about” is used synonymously with the term “approximately.” Illustratively, the use of the term “about” with regard to a certain therapeutically effective pharmaceutical dose indicates that values slightly outside the cited values, e.g., plus or minus 0.1% to 10%, are also effective and safe.

The term “patient”, “subject” or “individual” are used interchangeably. As used herein, they refer to individuals suffering from a disorder, and the like, encompasses mammals and non-mammals. None of the terms require that the individual be under the care and/or supervision of a medical professional. Mammals are any member of the Mammalian class, including but not limited to humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the individual is a mammal. In preferred embodiments, the individual is a human.

The terms “treat.” “treating” or “treatment,” and other grammatical equivalents as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The terms “administer,” “administering”, “administration,” and the like, as used herein, refer to the methods that may be used to enable delivery of compounds or compositions to the desired site of biological action. These methods include, but are not limited to topical routes, oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein.

The term “acceptable” as used herein, with respect to a formulation, composition or ingredient, means having no persistent detrimental effect on the general health of the individual being treated.

The term “pharmaceutically acceptable” as used herein, refers to a material, such as a carrier or diluent, which does not abrogate the biological activity or properties of the compounds described herein, and is relatively nontoxic, i.e., the material may be administered to an individual without causing undesirable biological effects or interacting in a deleterious manner with any of the components of the composition in which it is contained.

The term “DIM” as used herein, refers to a substituted or unsubstituted diindolylmethane compound.

The term “BR-DIM” or “BioResponse DIM” as used herein, refers to an unsubstituted DIM, provided by BioResponse LLC.

The term “maximum concentration” or Cmax as used herein, refers to the Cmax refers to the maximum (or peak) serum concentration that the substituted or unsubstituted diindolylmethane achieves in the plasma after it has been administrated and prior to the administration of a second dose.

The term “time to maximum concentration” or Tmax as used herein, refers to the time at which the Cmax is observed.

The term “area under the curve” or AUC or AUC_{0-inf} as used herein, refers to the area under the curve, also known as the definite integral, in a plot of concentration of drug in blood plasma against time.

The term “unit dose” as used herein, refers to an amount of substituted or unsubstituted diindolylmethane contained in one discreet pharmaceutical dosage form. Examples of pharmaceutical dosage form that contains a unit dose include but are not limited to a tablet, a capsule, a buccal tablet, a sub-lingual tablet, an orally-disintegrating tablet, an effervescent tablet, a lollipop, a lozenge, a troche, a liquid solution or suspension, powder or liquid or solid crystals packed within a single tablet or capsule, a cream, a gel, an ointment, a lotion.

### DIM

Diindolylmethane (DIM) is a natural compound formed during the autolytic breakdown of glucobrassicin present in food plants of the Brassica genus, including broccoli, cabbage, Brussels sprouts, cauliflower and kale. The autolytic breakdown of glucobrassicin requires the catalytic reaction of the enzyme myrosinase, which is endogenous to these plants and released upon rupture of the cell wall. The compound is normally manufactured by chemical synthesis but in some embodiments is also prepared by natural means from the extracts of Brassica vegetables, as listed above, particularly from sprouting broccoli or from broccoli seeds.

Thus, the substituted or unsubstituted DIM in some embodiments is synthetic, or in some embodiments is a natural product obtained from a Brassica plant, as discussed above.

In some embodiments, the composition described herein comprises a diindolylmethane of Formula 1:

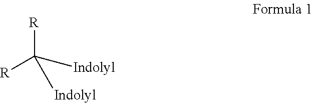

wherein the R groups are independently selected from hydrogen atoms and C1-C6 hydrocarbon substituents; and wherein the indolyl groups are independently selected from indole-3-yl and indole-2-yl groups; and wherein the indolyl groups are unsubstituted, or are substituted with one or more C1-C6 hydrocarbon substituents.

In some embodiments, the composition described herein comprises an unsubstituted DIM. In some embodiments, the composition described herein comprises an unsubstituted 3,3′diindolylmethane.

In some embodiments, the composition described herein comprises encapsulated DIM to further improve the bioavailability of DIM. In some embodiments, the encapsulated DIM is BR-DIM.

### Acne

Acne is a chronic inflammatory disease of the pilosebaceous unit resulting from androgen-induced increased sebum production, altered keratinisation, inflammation, and bacterial colonisation of hair follicles on the face, neck, chest, and back by Propionibacterium acnes. The initial pathology of acne is the comedo and includes acne vulgaris, neonatal acne, infantile acne, and pomade acne. The disease of acne is characterized by a great variety of clinical lesions. Although one type of lesion may be predominant (typically the comedo), close observation usually reveals the presence of several types of lesions (comedones, pustules, papules, and/or nodules). The lesions can be either noninflammatory or, more typically, inflammatory. In addition to lesions, patients may have, as the result of lesions, scars of varying size. The fully developed, open comedo (i.e., a plug of dried sebum in a skin pore) is not usually the site of inflammatory changes, unless it is traumatized by the patient. The developing microcomedo and the closed comedo are the major sites for the development of inflammatory lesions. Because the skin is always trying to repair itself, sheaths of cells will grow out from the epidermis (forming appendageal structures) in an attempt to encapsulate the inflammatory reaction. This encapsulation is often incomplete and further rupture of the lesion typically occurs, leading to multichanneled tracts as can be seen in many acne scars.

There are primarily four factors that are believed to be the contributors of acne: (1) Increased sebum production; (2) Comedo formation, in which the follicular infundibulum hypercornifies, hyperkeratinizes, and hypodesquamates; (3) Colonization of the follicule by anaerobic Propionibacterium, mainly P. acnes; and (4) The host's inflammatory response. These four factors are interrelated to each other. Sebum is comedogenic and causes inflammation by itself. The Propionibacterium has high lipolytic activity and liberates free fatty acids from sebum lipids. The free fatty acids have been shown to cause marked inflammation. The microorganisms also produce other extracellular enzymes such as proteases and hyaluronidases, and chemotactic factors, which may be important in the inflammatory process. Other factors such as diet have been implicated, but not proven. Facial scarring due to acne affects up to 20% of teenagers. Acne can persist into adulthood, with detrimental effects on self-esteem. The disease is so common in youth at their puberty that it often has been termed physiological. Although acne stops appearing for most people by the age of 25, some people, the majority of them are women, experience the disease well into their adult life. This “adult acne” differs from teenage acne in location and that it tends to be more inflammatory with fewer comedones.

In general, there are four major principles presently governing the therapy of acne: (i) correction of the altered pattern of follicular keratinization; (ii) decrease sebaceous gland activity; (iii) decrease the follicular bacterial population (especially P. acnes) and inhibit the production of extra cellular inflammatory products through the inhibition of these microorganisms; and (iv) produce an anti-inflammatory effect.

### Atopic Dermatitis

Atopic dermatitis, often referred to as eczema, is a chronic (long-lasting) disease that causes inflammation, redness, and irritation of the skin. It is a common condition that usually begins in childhood; however, anyone can get the disease at any age. Atopic dermatitis is not contagious, so it cannot be spread from person to person.

Atopic dermatitis causes the skin to become extremely itchy. Scratching leads to further redness, swelling, cracking, “weeping” clear fluid, crusting, and scaling. In most cases, there are periods of time when the disease is worse, called flares, followed by periods when the skin improves or clears up entirely, called remissions.

The exact underlying causes for atopic dermatitis is unknown. However, it is known that genes, the immune system, and the environment play a role in the disease. Depending on the severity and location of the symptoms, living with atopic dermatitis can be hard. Treatment can help control symptoms. For many people, atopic dermatitis improves by adulthood, but for some, it can be a lifelong illness.

### Rosacea

Rosacea is a chronic inflammatory condition of the facial skin affecting the blood vessels and pilosebaceous units. Rosacea is more common in persons of northern and western European descent with a fair complexion, but it can affect skin of any color. Although symptoms may wax and wane during the short term, rosacea can progress with time. Patients usually present with complaints of flushing and blushing and sensitive skin, and their skin may be especially irritated by topical preparations. Rosacea has a variety of triggers; however, they may be unnoticed by the patient.

It is a chronic and progressive cutaneous vascular disorder, primarily involving the malar and nasal areas of the face. Rosacea is characterized by flushing, erythema, papules, pustules, telanglectasia, facial edema, ocular lesions, and, in its most advanced and severe form, hyperplasia of tissue and sebaceous glands leading to rhinophyma. Rhinophyma, a florid overgrowth of the tip of the nose with hypervascularity and modularity, is an unusual progression of rosacea of unknown cause. Ocular lesions are common, including mild conjunctivitis, burning, and grittiness. Blepharitis, the most common ocular manifestation, is a nonulcerative condition of the lidmargins. Rosacea most commonly occurs between the ages of 30 to 60, and may be seen in women experiencing hormonal changes associated with menopause. Women are more frequently affected than men; the most severe cases, however, are seen in men.

### Skin Aging

Aging changes in the skin are a group of common conditions and developments that occur as people grow older. Skin changes are among the most visible signs of aging. Evidence of increasing age includes wrinkles and sagging skin. Whitening or graying of the hair is another obvious sign of aging.

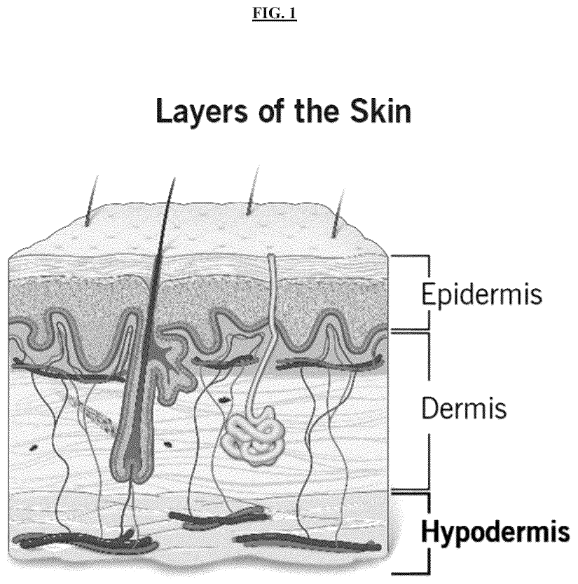

As shown in FIG. 1, skin has many layers and can generally be divided into three main parts: the outer part (epidermis) contains skin cells, pigment, and proteins; the middle part (dermis) contains skin cells, blood vessels, nerves, hair follicles, and oil glands; and the inner layer under the dermis (the subcutaneous layer or hypodermis) contains sweat glands, some hair follicles, blood vessels, and fat. Each layer also contains connective tissue with collagen fibers to give support and elastin fibers to provide flexibility and strength.

Skin changes are related to environmental factors, genetic makeup, nutrition, and other factors. The greatest single factor, though, is sun exposure. Natural pigments seem to provide some protection against sun-induced skin damage. Blue-eyed, fair-skinned people show more aging skin changes than people with darker, more heavily pigmented skin.

With aging, the outer skin layer (epidermis) thins, even though the number of cell layers remains unchanged. The number of pigment-containing cells (melanocytes) decreases. The remaining melanocytes increase in size. Aging skin looks thinner, paler, and clear (translucent). Pigmented spots including age spots or “liver spots” may appear in sun-exposed areas. The medical term for these areas is lentigos.

Changes in the connective tissue reduce the skin's strength and elasticity. This is known as elastosis. It is more noticeable in sun-exposed areas (solar elastosis). Elastosis produces the leathery, weather-beaten appearance common to farmers, sailors, and others who spend a large amount of time outdoors. The blood vessels of the dermis become more fragile. This leads to bruising, bleeding under the skin (often called senile purpura), and cherry angiomas. Sebaceous glands produce less oil as you age. Men experience a minimal decrease, most often after the age of 80. Women gradually produce less oil beginning after menopause. This can make it harder to keep the skin moist, resulting in dryness and itchiness.

The subcutaneous fat layer thins so it has less insulation and padding. This increases your risk of skin injury and reduces your ability to maintain body temperature. Because you have less natural insulation, you can get hypothermia in cold weather. Some medicines are absorbed by the fat layer. Shrinkage of this layer may change the way that these medicines work. The sweat glands produce less sweat. This makes it harder to keep cool. Your risk for overheating or developing heat stroke increases.

Growths such as skin tags, warts, brown rough patches (seborrheic keratoses), and other blemishes are more common in older people. Also common are pinkish rough patches (actinic keratosis) which have a small chance of becoming a skin cancer. Skin cancers are also common and usually located in sun-exposed areas.

### Methods of Treating Acne or Rosacea by Topical Administration of DIM

Provided herein in some embodiments are methods of treating acne or rosacea in a subject in need thereof comprising administering to the subject a composition comprising substituted or unsubstituted diindolylmethane, wherein the composition is administered by topical route. In some embodiments, the composition comprising substituted or unsubstituted diindolylmethane further comprises substituted or unsubstituted retinoic acid based component.

In some embodiments, topical delivery allows the substituted or unsubstituted diindolylmethane to dissolve in the immediate vicinity where the product is placed and then the drug enters directly into the blood stream, thereby increasing the dermal bioavailability of the substituted or unsubstituted diindolylmethane and exert its pharmacological effect rapidly.

In some embodiments, the increase in dermal bioavailability of substituted or unsubstituted diindolylmethane upon delivery by topical route is relative to the dermal bioavailability upon administration by oral route, at the same delivery dose, of the same composition. In some embodiments, the increase in dermal bioavailability of substituted or unsubstituted diindolylmethane upon delivery by topical route is relative to the dermal bioavailability upon administration by oral route, at the same delivery dose, of a comparative composition.

In some embodiments, administration of a composition comprising substituted or unsubstituted diindolylmethane by topical route helps by-pass the gastric juices, acid environment and enzymes present in the gastrointestinal tract, as compared to oral administration. In some embodiments, administration of a composition comprising substituted or unsubstituted diindolylmethane by topical route helps by-pass the saliva and enzymes present in the oral cavity, as compared to sublingual or buccal administration.

In some embodiments, increased dermal bioavailability of the substituted or unsubstituted diindolylmethane by topical administration is attributed to by-passing the gastric juices, acid environment and enzymes present in the gastrointestinal tract. In some embodiments, the administration of substituted or unsubstituted diindolylmethane by topical route helps by-pass the liver which is the target organ for metabolism of the drug when administered orally. In some embodiments, increased dermal bioavailability of the substituted or unsubstituted diindolylmethane by topical administration is attributed to by-passing first pass metabolism by the liver. In some embodiments, the highly vascular mucosal lining between the check and gum where buccal formulations are placed or under the tongue where sublingual formulations are placed are ideal and convenient locations for the substituted or unsubstituted diindolylmethane to be absorbed.

### Increase in Dermal Bioavailability of DIM by Topical Administration

In some embodiments, the administration by topical route increases the dermal bioavailability of the substituted or unsubstituted diindolylmethane by about 1-fold to about 1000-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 1-fold to about 500-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 1-fold to about 100-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 1-fold to about 50-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 1-fold to about 20-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 1-fold to about 10-fold compared to the dermal bioavailability of the substituted or unsubstituted diindolylmethane of a comparative composition administered by oral route at the same delivery dose.

In some embodiments, the administration by topical route increases the dermal bioavailability of the substituted or unsubstituted diindolylmethane by about 1-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 2-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 3-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 4-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 5-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 10-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 20-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 30-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 40-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 50-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 60-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 70-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 80-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 100-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 200-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 300-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 400-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 500-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 600-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 700-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 800-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 900-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 1000-fold compared to the dermal bioavailability of the substituted or unsubstituted diindolylmethane of a comparative composition administered by oral route at the same delivery dose. In some embodiments, the administration by topical route increases the dermal bioavailability of the substituted or unsubstituted diindolylmethane by about 100-fold compared to the dermal bioavailability of the substituted or unsubstituted diindolylmethane of a comparative composition administered by oral route at the same delivery dose.

### Improved Pharmacokinetic Parameters Upon Administration of DIM by Topical Route

Dermal bioavailability includes the following exemplary pharmacokinetic factors: rate (or time after administration) of achievement of minimum effective drug serum concentration (MEC), maximum drug serum concentration (Cmax), rate (or time after administration) of achievement of maximum drug serum concentration (Tmax), and the area under the drug serum concentration-time curve above a line representing minimum effective drug serum concentration (AUC). In some embodiments, the methods of treating a skin disease or condition by administration of substituted or unsubstituted diindolylmethane by topical route, as described herein, leads to enhancement in one more of the factors mentioned above.

### Increased Cmax

In some embodiments, following administration of the composition by topical route the Cmax of the substituted or unsubstituted diindolylmethane in skin increases by about 1-fold to about 1000-fold compared to the Cmax following administration of a comparative composition by oral route at the same delivery dose. In some embodiments, following administration of the composition by topical route the Cmax of the substituted or unsubstituted diindolylmethane in skin increases by about 1-fold to about 500-fold compared to the Cmax following administration of a comparative composition by oral route at the same delivery dose. In some embodiments, following administration of the composition by topical route the Cmax of the substituted or unsubstituted diindolylmethane in skin increases by about 1-fold to about 100-fold compared to the Cmax following administration of a comparative composition by oral route at the same delivery dose. In some embodiments, following administration of the composition by topical route the Cmax of the substituted or unsubstituted diindolylmethane in skin increases by about 1-fold to about 50-fold compared to the Cmax following administration of a comparative composition by oral route at the same delivery dose. In some embodiments, following administration of the composition by topical route the Cmax of the substituted or unsubstituted diindolylmethane in skin increases by about 1-fold to about 20-fold compared to the Cmax following administration of a comparative composition by oral route at the same delivery dose. In some embodiments, following administration of the composition by topical route the Cmax of the substituted or unsubstituted diindolylmethane in skin increases by about 1-fold to about 10-fold compared to the Cmax following administration of a comparative composition by oral route at the same delivery dose.

In some embodiments, following administration of the composition by topical route the Cmax of the substituted or unsubstituted diindolylmethane in skin increases by about 1-fold compared to the Cmax following administration of a comparative composition by oral route at the same delivery dose. In some embodiments, following administration of the composition by topical route the Cmax of the substituted or unsubstituted diindolylmethane in skin increases by about 2-fold compared to the Cmax following administration of a comparative composition by oral route at the same delivery dose. In some embodiments, following administration of the composition by topical route the Cmax of the substituted or unsubstituted diindolylmethane in skin increases by about 3-fold compared to the Cmax following administration of a comparative composition by oral route at the same delivery dose. In some embodiments, following administration of the composition by topical route the Cmax of the substituted or unsubstituted diindolylmethane in skin increases by about 4-fold compared to the Cmax following administration of a comparative composition by oral route at the same delivery dose. In some embodiments, following administration of the composition by topical route the Cmax of the substituted or unsubstituted diindolylmethane in skin increases by about 5-fold compared to the Cmax following administration of a comparative composition by oral route at the same delivery dose. In some embodiments, following administration of the composition by topical route the Cmax of the substituted or unsubstituted diindolylmethane in skin increases by about 6-fold compared to the Cmax following administration of a comparative composition by oral route at the same delivery dose. In some embodiments, following administration of the composition by topical route the Cmax of the substituted or unsubstituted diindolylmethane in skin increases by about 7-fold compared to the Cmax following administration of a comparative composition by oral route at the same delivery dose. In some embodiments, following administration of the composition by topical route the Cmax of the substituted or unsubstituted diindolylmethane in skin increases by about 8-fold compared to the Cmax following administration of a comparative composition by oral route at the same delivery dose. In some embodiments, following administration of the composition by topical route the Cmax of the substituted or unsubstituted diindolylmethane in skin increases by about 9-fold compared to the Cmax following administration of a comparative composition by oral route at the same delivery dose. In some embodiments, following administration of the composition by topical route the Cmax of the substituted or unsubstituted diindolylmethane in skin increases by about 10-fold compared to the Cmax following administration of a comparative composition by oral route at the same delivery dose. In some embodiments, following administration of the composition by topical route the Cmax of the substituted or unsubstituted diindolylmethane in skin increases by about 20-fold compared to the Cmax following administration of a comparative composition by oral route at the same delivery dose. In some embodiments, following administration of the composition by topical route the Cmax of the substituted or unsubstituted diindolylmethane in skin increases by about 30-fold compared to the Cmax following administration of a comparative composition by oral route at the same delivery dose. In some embodiments, following administration of the composition by topical route the Cmax of the substituted or unsubstituted diindolylmethane in skin increases by about 40-fold compared to the Cmax following administration of a comparative composition by oral route at the same delivery dose. In some embodiments, following administration of the composition by topical route the Cmax of the substituted or unsubstituted diindolylmethane in skin increases by about 50-fold compared to the Cmax following administration of a comparative composition by oral route at the same delivery dose. In some embodiments, following administration of the composition by topical route the Cmax of the substituted or unsubstituted diindolylmethane in skin increases by about 60-fold compared to the Cmax following administration of a comparative composition by oral route at the same delivery dose. In some embodiments, following administration of the composition by topical route the Cmax of the substituted or unsubstituted diindolylmethane in skin increases by about 70-fold compared to the Cmax following administration of a comparative composition by oral route at the same delivery dose. In some embodiments, following administration of the composition by topical route the Cmax of the substituted or unsubstituted diindolylmethane in skin increases by about 80-fold compared to the Cmax following administration of a comparative composition by oral route at the same delivery dose. In some embodiments, following administration of the composition by topical route the Cmax of the substituted or unsubstituted diindolylmethane in skin increases by about 90-fold compared to the Cmax following administration of a comparative composition by oral route at the same delivery dose. In some embodiments, following administration of the composition by topical route the Cmax of the substituted or unsubstituted diindolylmethane in skin increases by about 100-fold compared to the Cmax following administration of a comparative composition by oral route at the same delivery dose. In some embodiments, following administration of the composition by topical route the Cmax of the substituted or unsubstituted diindolylmethane in skin increases by about 200-fold compared to the Cmax following administration of a comparative composition by oral route at the same delivery dose. In some embodiments, following administration of the composition by topical route the Cmax of the substituted or unsubstituted diindolylmethane in skin increases by about 300-fold compared to the Cmax following administration of a comparative composition by oral route at the same delivery dose. In some embodiments, following administration of the composition by topical route the Cmax of the substituted or unsubstituted diindolylmethane in skin increases by about 400-fold compared to the Cmax following administration of a comparative composition by oral route at the same delivery dose. In some embodiments, following administration of the composition by topical route the Cmax of the substituted or unsubstituted diindolylmethane in skin increases by about 500-fold compared to the Cmax following administration of a comparative composition by oral route at the same delivery dose. In some embodiments, following administration of the composition by topical route the Cmax of the substituted or unsubstituted diindolylmethane in skin increases by about 600-fold compared to the Cmax following administration of a comparative composition by oral route at the same delivery dose. In some embodiments, following administration of the composition by topical route the Cmax of the substituted or unsubstituted diindolylmethane in skin increases by about 700-fold compared to the Cmax following administration of a comparative composition by oral route at the same delivery dose. In some embodiments, following administration of the composition by topical route the Cmax of the substituted or unsubstituted diindolylmethane in skin increases by about 800-fold compared to the Cmax following administration of a comparative composition by oral route at the same delivery dose. In some embodiments, following administration of the composition by topical route the Cmax of the substituted or unsubstituted diindolylmethane in skin increases by about 800-fold compared to the Cmax following administration of a comparative composition by oral route at the same delivery dose. In some embodiments, following administration of the composition by topical route the Cmax of the substituted or unsubstituted diindolylmethane in skin increases by about 900-fold compared to the Cmax following administration of a comparative composition by oral route at the same delivery dose. In some embodiments, following administration of the composition by topical route the Cmax of the substituted or unsubstituted diindolylmethane in skin increases by about 1000-fold compared to the Cmax following administration of a comparative composition by oral route at the same delivery dose.

### Increased AUC

In some embodiments, following administration of the composition by topical route the AUC of the substituted or unsubstituted diindolylmethane in skin increases by about 1-fold to about 1000-fold compared to the AUC following administration of a comparative composition by oral route at the same delivery dose. In some embodiments, following administration of the composition by topical route the AUC of the substituted or unsubstituted diindolylmethane in skin increases by about 1-fold to about 500-fold compared to the AUC following administration of a comparative composition by oral route at the same delivery dose. In some embodiments, following administration of the composition by topical route the AUC of the substituted or unsubstituted diindolylmethane in skin increases by about 1-fold to about 100-fold compared to the AUC following administration of a comparative composition by oral route at the same delivery dose. In some embodiments, following administration of the composition by topical route the AUC of the substituted or unsubstituted diindolylmethane in skin increases by about 1-fold to about 50-fold compared to the AUC following administration of a comparative composition by oral route at the same delivery dose. In some embodiments, following administration of the composition by topical route the AUC of the substituted or unsubstituted diindolylmethane in skin increases by about 1-fold to about 20-fold compared to the AUC following administration of a comparative composition by oral route at the same delivery dose. In some embodiments, following administration of the composition by topical route the AUC of the substituted or unsubstituted diindolylmethane in skin increases by about 1-fold to about 10-fold compared to the AUC following administration of a comparative composition by oral route at the same delivery dose.

In some embodiments, following administration of the composition by topical route the AUC of the substituted or unsubstituted diindolylmethane in skin increases by about 1-fold compared to the AUC following administration of a comparative composition by oral route at the same delivery dose. In some embodiments, following administration of the composition by topical route the AUC of the substituted or unsubstituted diindolylmethane in skin increases by about 2-fold compared to the AUC following administration of a comparative composition by oral route at the same delivery dose. In some embodiments, following administration of the composition by topical route the AUC of the substituted or unsubstituted diindolylmethane in skin increases by about 3-fold compared to the AUC following administration of a comparative composition by oral route at the same delivery dose. In some embodiments, following administration of the composition by topical route the AUC of the substituted or unsubstituted diindolylmethane in skin increases by about 4-fold compared to the AUC following administration of a comparative composition by oral route at the same delivery dose. In some embodiments, following administration of the composition by topical route the AUC of the substituted or unsubstituted diindolylmethane in skin increases by about 5-fold compared to the AUC following administration of a comparative composition by oral route at the same delivery dose. In some embodiments, following administration of the composition by topical route the AUC of the substituted or unsubstituted diindolylmethane in skin increases by about 6-fold compared to the AUC following administration of a comparative composition by oral route at the same delivery dose. In some embodiments, following administration of the composition by topical route the AUC of the substituted or unsubstituted diindolylmethane in skin increases by about 7-fold compared to the AUC following administration of a comparative composition by oral route at the same delivery dose. In some embodiments, following administration of the composition by topical route the AUC of the substituted or unsubstituted diindolylmethane in skin increases by about 8-fold compared to the AUC following administration of a comparative composition by oral route at the same delivery dose. In some embodiments, following administration of the composition by topical route the AUC of the substituted or unsubstituted diindolylmethane in skin increases by about 9-fold compared to the AUC following administration of a comparative composition by oral route at the same delivery dose. In some embodiments, following administration of the composition by topical route the AUC of the substituted or unsubstituted diindolylmethane in skin increases by about 10-fold compared to the AUC following administration of a comparative composition by oral route at the same delivery dose. In some embodiments, following administration of the composition by topical route the AUC of the substituted or unsubstituted diindolylmethane in skin increases by about 20-fold compared to the AUC following administration of a comparative composition by oral route at the same delivery dose. In some embodiments, following administration of the composition by topical route the AUC of the substituted or unsubstituted diindolylmethane in skin increases by about 30-fold compared to the AUC following administration of a comparative composition by oral route at the same delivery dose. In some embodiments, following administration of the composition by topical route the AUC of the substituted or unsubstituted diindolylmethane in skin increases by about 40-fold compared to the AUC following administration of a comparative composition by oral route at the same delivery dose. In some embodiments, following administration of the composition by topical route the AUC of the substituted or unsubstituted diindolylmethane in skin increases by about 50-fold compared to the AUC following administration of a comparative composition by oral route at the same delivery dose. In some embodiments, following administration of the composition by topical route the AUC of the substituted or unsubstituted diindolylmethane in skin increases by about 60-fold compared to the AUC following administration of a comparative composition by oral route at the same delivery dose. In some embodiments, following administration of the composition by topical route the AUC of the substituted or unsubstituted diindolylmethane in skin increases by about 70-fold compared to the AUC following administration of a comparative composition by oral route at the same delivery dose. In some embodiments, following administration of the composition by topical route the AUC of the substituted or unsubstituted diindolylmethane in skin increases by about 80-fold compared to the AUC following administration of a comparative composition by oral route at the same delivery dose. In some embodiments, following administration of the composition by topical route the AUC of the substituted or unsubstituted diindolylmethane in skin increases by about 90-fold compared to the AUC following administration of a comparative composition by oral route at the same delivery dose. In some embodiments, following administration of the composition by topical route the AUC of the substituted or unsubstituted diindolylmethane in skin increases by about 100-fold compared to the AUC following administration of a comparative composition by oral route at the same delivery dose. In some embodiments, following administration of the composition by topical route the AUC of the substituted or unsubstituted diindolylmethane in skin increases by about 200-fold compared to the AUC following administration of a comparative composition by oral route at the same delivery dose. In some embodiments, following administration of the composition by topical route the AUC of the substituted or unsubstituted diindolylmethane in skin increases by about 300-fold compared to the AUC following administration of a comparative composition by oral route at the same delivery dose. In some embodiments, following administration of the composition by topical route the AUC of the substituted or unsubstituted diindolylmethane in skin increases by about 400-fold compared to the AUC following administration of a comparative composition by oral route at the same delivery dose. In some embodiments, following administration of the composition by topical route the AUC of the substituted or unsubstituted diindolylmethane in skin increases by about 500-fold compared to the AUC following administration of a comparative composition by oral route at the same delivery dose. In some embodiments, following administration of the composition by topical route the AUC of the substituted or unsubstituted diindolylmethane in skin increases by about 600-fold compared to the AUC following administration of a comparative composition by oral route at the same delivery dose. In some embodiments, following administration of the composition by topical route the AUC of the substituted or unsubstituted diindolylmethane in skin increases by about 700-fold compared to the AUC following administration of a comparative composition by oral route at the same delivery dose. In some embodiments, following administration of the composition by topical route the AUC of the substituted or unsubstituted diindolylmethane in skin increases by about 800-fold compared to the AUC following administration of a comparative composition by oral route at the same delivery dose. In some embodiments, following administration of the composition by topical route the AUC of the substituted or unsubstituted diindolylmethane in skin increases by about 800-fold compared to the AUC following administration of a comparative composition by oral route at the same delivery dose. In some embodiments, following administration of the composition by topical route the AUC of the substituted or unsubstituted diindolylmethane in skin increases by about 900-fold compared to the AUC following administration of a comparative composition by oral route at the same delivery dose. In some embodiments, following administration of the composition by topical route the AUC of the substituted or unsubstituted diindolylmethane in skin increases by about 1000-fold compared to the AUC following administration of a comparative composition by oral route at the same delivery dose.

### Decreased Tmax

In some embodiments, following administration of the composition by topical route the Tmax of the substituted or unsubstituted diindolylmethane in skin decreases by about 1-fold to about 1000-fold compared to the Tmax following administration of a comparative composition by oral route at the same delivery dose. In some embodiments, following administration of the composition by topical route the Tmax of the substituted or unsubstituted diindolylmethane in skin decreases by about 1-fold to about 500-fold compared to the Tmax following administration of a comparative composition by oral route at the same delivery dose. In some embodiments, following administration of the composition by topical route the Tmax of the substituted or unsubstituted diindolylmethane in skin decreases by about 1-fold to about 100-fold compared to the Tmax following administration of a comparative composition by oral route at the same delivery dose. In some embodiments, following administration of the composition by topical route the Tmax of the substituted or unsubstituted diindolylmethane in skin decreases by about 1-fold to about 50-fold compared to the Tmax following administration of a comparative composition by oral route at the same delivery dose. In some embodiments, following administration of the composition by topical route the Tmax of the substituted or unsubstituted diindolylmethane in skin decreases by about 1-fold to about 20-fold compared to the Tmax following administration of a comparative composition by oral route at the same delivery dose. In some embodiments, following administration of the composition by topical route the Tmax of the substituted or unsubstituted diindolylmethane in skin decreases by about 1-fold to about 10-fold compared to the Tmax following administration of a comparative composition by oral route at the same delivery dose.

In some embodiments, following administration of the composition by topical route the Tmax of the substituted or unsubstituted diindolylmethane in skin decreases by about 1-fold compared to the Tmax following administration of a comparative composition by oral route at the same delivery dose. In some embodiments, following administration of the composition by topical route the Tmax of the substituted or unsubstituted diindolylmethane in skin decreases by about 2-fold compared to the Tmax following administration of a comparative composition by oral route at the same delivery dose. In some embodiments, following administration of the composition by topical route the Tmax of the substituted or unsubstituted diindolylmethane in skin decreases by about 3-fold compared to the Tmax following administration of a comparative composition by oral route at the same delivery dose. In some embodiments, following administration of the composition by topical route the Tmax of the substituted or unsubstituted diindolylmethane in skin decreases by about 4-fold compared to the Tmax following administration of a comparative composition by oral route at the same delivery dose. In some embodiments, following administration of the composition by topical route the Tmax of the substituted or unsubstituted diindolylmethane in skin decreases by about 5-fold compared to the Tmax following administration of a comparative composition by oral route at the same delivery dose. In some embodiments, following administration of the composition by topical route the Tmax of the substituted or unsubstituted diindolylmethane in skin decreases by about 6-fold compared to the Tmax following administration of a comparative composition by oral route at the same delivery dose. In some embodiments, following administration of the composition by topical route the Tmax of the substituted or unsubstituted diindolylmethane in skin decreases by about 7-fold compared to the Tmax following administration of a comparative composition by oral route at the same delivery dose. In some embodiments, following administration of the composition by topical route the Tmax of the substituted or unsubstituted diindolylmethane in skin decreases by about 8-fold compared to the Tmax following administration of a comparative composition by oral route at the same delivery dose. In some embodiments, following administration of the composition by topical route the Tmax of the substituted or unsubstituted diindolylmethane in skin decreases by about 9-fold compared to the Tmax following administration of a comparative composition by oral route at the same delivery dose. In some embodiments, following administration of the composition by topical route the Tmax of the substituted or unsubstituted diindolylmethane in skin decreases by about 10-fold compared to the Tmax following administration of a comparative composition by oral route at the same delivery dose. In some embodiments, following administration of the composition by topical route the Tmax of the substituted or unsubstituted diindolylmethane in skin decreases by about 20-fold compared to the Tmax following administration of a comparative composition by oral route at the same delivery dose. In some embodiments, following administration of the composition by topical route the Tmax of the substituted or unsubstituted diindolylmethane in skin decreases by about 30-fold compared to the Tmax following administration of a comparative composition by oral route at the same delivery dose. In some embodiments, following administration of the composition by topical route the Tmax of the substituted or unsubstituted diindolylmethane in skin decreases by about 40-fold compared to the Tmax following administration of a comparative composition by oral route at the same delivery dose. In some embodiments, following administration of the composition by topical route the Tmax of the substituted or unsubstituted diindolylmethane in skin decreases by about 50-fold compared to the Tmax following administration of a comparative composition by oral route at the same delivery dose. In some embodiments, following administration of the composition by topical route the Tmax of the substituted or unsubstituted diindolylmethane in skin decreases by about 60-fold compared to the Tmax following administration of a comparative composition by oral route at the same delivery dose. In some embodiments, following administration of the composition by topical route the Tmax of the substituted or unsubstituted diindolylmethane in skin decreases by about 70-fold compared to the Tmax following administration of a comparative composition by oral route at the same delivery dose. In some embodiments, following administration of the composition by topical route the Tmax of the substituted or unsubstituted diindolylmethane in skin decreases by about 80-fold compared to the Tmax following administration of a comparative composition by oral route at the same delivery dose. In some embodiments, following administration of the composition by topical route the Tmax of the substituted or unsubstituted diindolylmethane in skin decreases by about 100-fold compared to the Tmax following administration of a comparative composition by oral route at the same delivery dose. In some embodiments, following administration of the composition by topical route the Tmax of the substituted or unsubstituted diindolylmethane in skin decreases by about 200-fold compared to the Tmax following administration of a comparative composition by oral route at the same delivery dose. In some embodiments, following administration of the composition by topical route the Tmax of the substituted or unsubstituted diindolylmethane in skin decreases by about 300-fold compared to the Tmax following administration of a comparative composition by oral route at the same delivery dose. In some embodiments, following administration of the composition by topical route the Tmax of the substituted or unsubstituted diindolylmethane in skin decreases by about 400-fold compared to the Tmax following administration of a comparative composition by oral route at the same delivery dose. In some embodiments, following administration of the composition by topical route the Tmax of the substituted or unsubstituted diindolylmethane in skin decreases by about 500-fold compared to the Tmax following administration of a comparative composition by oral route at the same delivery dose. In some embodiments, following administration of the composition by topical route the Tmax of the substituted or unsubstituted diindolylmethane in skin decreases by about 600-fold compared to the Tmax following administration of a comparative composition by oral route at the same delivery dose. In some embodiments, following administration of the composition by topical route the Tmax of the substituted or unsubstituted diindolylmethane in skin decreases by about 700-fold compared to the Tmax following administration of a comparative composition by oral route at the same delivery dose. In some embodiments, following administration of the composition by topical route the Tmax of the substituted or unsubstituted diindolylmethane in skin decreases by about 800-fold compared to the Tmax following administration of a comparative composition by oral route at the same delivery dose. In some embodiments, following administration of the composition by topical route the Tmax of the substituted or unsubstituted diindolylmethane in skin decreases by about 900-fold compared to the Tmax following administration of a comparative composition by oral route at the same delivery dose. In some embodiments, following administration of the composition by topical route the Tmax of the substituted or unsubstituted diindolylmethane in skin decreases by about 1000-fold compared to the Tmax following administration of a comparative composition by oral route at the same delivery dose.

### DIM Composition, Formulations, Routes of Administration and Delivery Dosage

In some embodiments, the compositions described herein comprise a substituted or unsubstituted diindolylmethane. In some embodiments, the compositions described herein comprise a substituted or unsubstituted diindolylmethane that has been adapted to improve dermal bioavailability. In some embodiments, the substituted or unsubstituted diindolylmethane that has been adapted to improve dermal bioavailability is Bioresponse-diindolylmethane (BR-DIM). In some embodiments, the composition described herein comprises substituted or unsubstituted diindolylmethane that has not been adapted for improved dermal bioavailability. In some embodiments, the substituted or unsubstituted diindolylmethane that has not been adapted for improved dermal bioavailability is not BR-DIM. In some embodiments, the compositions described herein further comprise a substituted or unsubstituted retinoic acid based component.

In some embodiments, the retinoic acid based component is any such compound known in the art that is suitable for topical, oral, or topical administration. For example, in some embodiments, it is selected from a substituted or unsubstituted first generation retinoid, a substituted or unsubstituted second generation retinoid, and a substituted or unsubstituted third generation retinoid. In some embodiments, the retinoid is a substituted or unsubstituted first generation retinoid. In some embodiments, the substituted or unsubstituted first generation retinoid is selected from a substituted or unsubstituted retinol, a substituted or unsubstituted retinal, a substituted or unsubstituted tretinoin (e.g., retinoic acid or Retin A), a substituted or unsubstituted isotretinoin (e.g., Accutane™), and a substituted or unsubstituted alitretinoin. In some embodiments, the retinoid is vitamin A. In some embodiments, the retinoid is a substituted or unsubstituted second generation retinoid selected from a substituted or unsubstituted etretinate, and a substituted or unsubstituted acitretin. In some embodiments, the retinoid is a substituted or unsubstituted third generation retinoid selected from a substituted or unsubstituted tazarotene, a substituted or unsubstituted bexarotene, and a substituted or unsubstituted adapalene.

In some embodiments the composition comprising substituted or unsubstituted diindolylmethane is administered at a delivery dose that is sufficiently low to avoid toxicity, whilst still maintaining the required pharmaceutical effect. In some embodiments, the delivery dose of the composition varies depending upon whether it is a natural or synthetic product. In some embodiments, the delivery dose of the composition comprising the substituted or unsubstituted diindolylmethane is determined by whether or not the diindolylmethane has been adapted to improve dermal bioavailability. In some embodiments, the delivery dose for a composition comprising DIM is less than the delivery dose for a composition comprising substituted or unsubstituted diindolylmethane that has not been adapted to improve dermal bioavailability.

In some embodiments, the delivery dose is from about 0.01 mg to about 100 mg, from about 0.01 mg to about 75 mg, from about 0.01 mg to about 50 mg, from about 0.01 mg to about 25 mg, from about 0.01 mg to about 20 mg, from about 0.01 mg to about 15 mg, from about 0.01 mg to about 14 mg, from about 0.01 mg to about 13 mg, from about 0.01 mg to about 12 mg, from about 0.01 mg to about 11 mg, from about 0.01 mg to about 10 mg, from about 0.01 mg to about 9 mg, from about 0.01 mg to about 8 mg, from about 0.01 mg to about 7 mg, from about 0.01 mg to about 6 mg, from about 0.01 mg to about 9 mg, from about 0.01 mg to about 5 mg, from about 0.01 mg to about 4 mg, from about 0.01 mg to about 3 mg, from about 0.01 mg to about 2 mg, or from about 0.01 mg to about 1 mg.

In some embodiments, the delivery dose is from about 0.1 mg to about 100 mg, from about 0.1 mg to about 75 mg, from about 0.1 mg to about 50 mg, from about 0.1 mg to about 25 mg, from about 0.1 mg to about 20 mg, from about 0.1 mg to about 15 mg, from about 0.1 mg to about 14 mg, from about 0.1 mg to about 13 mg, from about 0.1 mg to about 12 mg, from about 0.1 mg to about 11 mg, from about 0.1 mg to about 10 mg, from about 0.1 mg to about 9 mg, from about 0.1 mg to about 8 mg, from about 0.1 mg to about 7 mg, from about 0.1 mg to about 6 mg, from about 0.1 mg to about 9 mg, from about 0.1 mg to about 5 mg, from about 0.1 mg to about 4 mg, from about 0.1 mg to about 3 mg, from about 0.1 mg to about 2 mg, or from about 0.1 mg to about 1 mg.

In some embodiments, the delivery dose is from about 0.2 mg to about 100 mg, from about 0.2 mg to about 75 mg, from about 0.2 mg to about 50 mg, from about 0.2 mg to about 25 mg, from about 0.2 mg to about 20 mg, from about 0.2 mg to about 15 mg, from about 0.2 mg to about 14 mg, from about 0.2 mg to about 13 mg, from about 0.2 mg to about 12 mg, from about 0.2 mg to about 11 mg, from about 0.2 mg to about 10 mg, from about 0.2 mg to about 9 mg, from about 0.2 mg to about 8 mg, from about 0.2 mg to about 7 mg, from about 0.2 mg to about 6 mg, from about 0.2 mg to about 9 mg, from about 0.2 mg to about 5 mg, from about 0.2 mg to about 4 mg, from about 0.2 mg to about 3 mg, from about 0.2 mg to about 2 mg, or from about 0.2 mg to about 1 mg.

In some embodiments, the delivery dose is from about 0.3 mg to about 100 mg, from about 0.3 mg to about 75 mg, from about 0.3 mg to about 50 mg, from about 0.3 mg to about 25 mg, from about 0.3 mg to about 20 mg, from about 0.3 mg to about 15 mg, from about 0.3 mg to about 14 mg, from about 0.3 mg to about 13 mg, from about 0.3 mg to about 12 mg, from about 0.3 mg to about 11 mg, from about 0.3 mg to about 10 mg, from about 0.3 mg to about 9 mg, from about 0.3 mg to about 8 mg, from about 0.3 mg to about 7 mg, from about 0.3 mg to about 6 mg, from about 0.3 mg to about 9 mg, from about 0.3 mg to about 5 mg, from about 0.3 mg to about 4 mg, from about 0.3 mg to about 3 mg, from about 0.3 mg to about 2 mg, or from about 0.3 mg to about 1 mg.

In some embodiments, the delivery dose is from about 0.4 mg to about 100 mg, from about 0.4 mg to about 75 mg, from about 0.4 mg to about 50 mg, from about 0.4 mg to about 25 mg, from about 0.4 mg to about 20 mg, from about 0.4 mg to about 15 mg, from about 0.4 mg to about 14 mg, from about 0.4 mg to about 13 mg, from about 0.4 mg to about 12 mg, from about 0.4 mg to about 11 mg, from about 0.4 mg to about 10 mg, from about 0.4 mg to about 9 mg, from about 0.4 mg to about 8 mg, from about 0.4 mg to about 7 mg, from about 0.4 mg to about 6 mg, from about 0.4 mg to about 9 mg, from about 0.4 mg to about 5 mg, from about 0.4 mg to about 4 mg, from about 0.4 mg to about 3 mg, from about 0.4 mg to about 2 mg, or from about 0.4 mg to about 1 mg.

In some embodiments, the delivery dose is from about 0.5 mg to about 100 mg, from about 0.5 mg to about 75 mg, from about 0.5 mg to about 50 mg, from about 0.5 mg to about 25 mg, from about 0.5 mg to about 20 mg, from about 0.5 mg to about 15 mg, from about 0.5 mg to about 14 mg, from about 0.5 mg to about 13 mg, from about 0.5 mg to about 12 mg, from about 0.5 mg to about 11 mg, from about 0.5 mg to about 10 mg, from about 0.5 mg to about 9 mg, from about 0.5 mg to about 8 mg, from about 0.5 mg to about 7 mg, from about 0.5 mg to about 6 mg, from about 0.5 mg to about 9 mg, from about 0.5 mg to about 5 mg, from about 0.5 mg to about 4 mg, from about 0.5 mg to about 3 mg, from about 0.5 mg to about 2 mg, or from about 0.5 mg to about 1 mg.

In some embodiments, the delivery dose of the compositions described herein provides a daily dose of the substituted or unsubstituted diindolylmethane from about 10 mg to about 20 mg, from about 15 mg to about 25 mg, from about 20 mg to about 30 mg, from about 25 mg to about 35 mg, from about 30 mg to about 40 mg, from about 35 mg to about 45 mg, from about 40 mg to about 50 mg, from about 45 mg to about 55 mg, from about 50 mg to about 100 mg, from about 55 mg to about 150 mg, from about 60 mg to about 200 mg, from about 65 mg to about 250 mg, from about 70 mg to about 300 mg.

In some embodiments, the delivery dose of the compositions described herein provides a daily dose of substituted or unsubstituted diindolylmethane that is at least 20 mg, at least 30 mg, at least 40 mg, at least 55 mg, at least 65 mg, at least 75 mg, at least 90 mg, at least 100 mg, at least 110 mg, at least 120 mg, at least 130 mg, at least 140 mg, or at least 150 mg.

In some embodiments, the composition comprising a substituted or unsubstituted diindolylmethane is administered in any of the above dosages, including the higher dosages, if desired. In some embodiments, the composition comprising a substituted or unsubstituted diindolylmethane that has been adapted to improve dermal bioavailability is administered in any of the above dosages, including the higher dosages, if desired. In some embodiments, the substituted or unsubstituted diindolylmethane that has been adapted to improve dermal bioavailability is BR-DIM.

In some embodiments, the composition comprising substituted or unsubstituted diinolylmethane is a formulation which is administered by topical route. The term “formulation which is administered by topical route” as used herein refers to a drug delivery formulation wherein an active compound is provided for penetration into and/or absorption by tissues and cells of various skin layers, such as epidermis, dermis, and/or hypodermis. The term encompasses suitable dosage forms, including gels, creams, ointments, liquids or viscous liquids (standalone or impregnated in a substrate, matrix or sponge), transdermal patches, bandages, etc. The standalone liquids or viscous liquids can be a solution, emulsion or a suspension.

In some embodiments, a comparative composition comprising substituted or unsubstituted diinolylmethane is a formulation which is administered by oral route. In some embodiments, the formulation which is administered by oral route is in the form of a tablet, capsule, or powder.

In some embodiments, a comparative composition comprising substituted or unsubstituted diinolylmethane is a formulation which is administered by sublingual or bucaal route. In some embodiments, the formulation which is administered by oral route is in the form of a tablet, lonzenge, or film.

In some embodiments the delivery dose for topical administration is no more than 15 mg. In some embodiments the delivery dose for topical administration is no more than 14 mg. In some embodiments the delivery dose for topical administration is no more than 13 mg. In some embodiments the delivery dose for topical administration is no more than 12 mg. In some embodiments the delivery dose for topical administration is no more than 11 mg. In some embodiments the delivery dose for topical administration is no more than 10 mg. In some embodiments the delivery dose for topical administration is no more than 9 mg. In some embodiments the delivery dose for topical administration is no more than 8 mg. In some embodiments the delivery dose for topical administration is no more than 7 mg. In some embodiments the delivery dose for topical administration is no more than 6 mg. In some embodiments the delivery dose for topical administration is no more than 5 mg. In some embodiments the delivery dose for topical administration is no more than 4 mg. In some embodiments the delivery dose for topical administration is no more than 3 mg. In some embodiments the delivery dose for topical administration is no more than 2 mg. In some embodiments the delivery dose for topical administration is no more than 1 mg. In some embodiments the delivery dose for topical administration is no more than 0.5 mg.

In some embodiments the delivery dose for topical administration is about 15 mg. In some embodiments the delivery dose for topical administration is about 14 mg. In some embodiments the delivery dose for topical administration is about 13 mg. In some embodiments the delivery dose for topical administration is about 12 mg. In some embodiments the delivery dose for topical administration is about 11 mg. In some embodiments the delivery dose for topical administration is about 10 mg. In some embodiments the delivery dose for topical administration is about 9 mg. In some embodiments the delivery dose for topical administration is about 8 mg. In some embodiments the delivery dose for topical administration is about 7 mg. In some embodiments the delivery dose for topical administration is about 6 mg. In some embodiments the delivery dose for topical administration is about 5 mg. In some embodiments the delivery dose for topical administration is about 4 mg. In some embodiments the delivery dose for topical administration is about 3 mg. In some embodiments the delivery dose for topical administration is about 2 mg. In some embodiments the delivery dose for topical administration is about 1 mg. In some embodiments the delivery dose for topical administration is about 0.5 mg.

In some embodiments, the delivery doses for administration by topical route and by oral route are the same. In some embodiments, the delivery dose for administration by topical route is less than the delivery dose for administration by oral route.

In some embodiments, the compositions described herein comprise substituted or unsubstituted diindolylmethane in a dose which is a fraction of the daily dose, such as a half of the daily dose, or a quarter of the daily dose, and thus is present in a half or a quarter of any of the dosages recited above. In these embodiments, each dose fraction is taken separately over time to spread the dose across the day.

In some embodiments, the compositions described herein further comprise a substituted or unsubstituted retinoid compound (e.g. retinoic acid, vitamin C, retinyl palmitate). In some embodiments, the retinoid compound is administered at a dosage that it is sufficiently low to avoid toxicity, whilst still maintaining the required pharmaceutical effect. In some embodiments, the delivery dosage of the retinoic compound depends on the dermal bioavailability of the same. In some embodiments, the dermal bioavailability of the substituted or unsubstituted retinoic compound varies depending upon whether it is a natural or synthetic product. In some embodiments, the dermal bioavailability of the substituted or unsubstituted retinoic compound may vary depending on whether it has been adapted to improve its dermal bioavailability.

In some embodiments, daily dosage of the composition comprising a substituted or unsubstituted diindolylmethane and optionally a substituted or unsubstituted retinoic compound is provided in the form of one or more unit doses In some embodiments, daily dosage of the composition comprising a substituted or unsubstituted diindolylmethane and optionally a substituted or unsubstituted retinoic acid based component is provided in the form of 2 to 4 unit doses. In these embodiments the two or more unit doses are taken during the course of a single day, such as one unit dose in the morning and one unit dose in the evening, or four unit doses spread evenly across the day, or two unit doses simultaneously twice a day.

### Methods for Preparation

In some embodiments, are provided, methods for preparing the compositions described herein, for use in the methods described herein, of treating acne or rosacea. In some embodiments, using the methods described herein, a composition suitable for topical administration is prepared. In some embodiments, using the methods described herein, a comparative composition suitable for oral administration is prepared.

In some embodiments, any methods known in the art for blending or mixing various components of the composition are employed. In some embodiments, the methods employed are methods for blending and/or mixing powders or fluids. In some embodiments, the method comprises mixing substituted or unsubstituted diindolylmethane with one or more pharmaceutically acceptable excipients and/or additives, to form the composition. In some embodiments, the substituted or unsubstituted diindolylmethane and the one or more pharmaceutically acceptable excipients and/or additives are each, separately from each other, mixed with one or more pharmaceutically acceptable excipients and/or additives before being mixed together to form the composition. In some embodiments, the substituted or unsubstituted diindolylmethane and the pharmaceutically acceptable excipients are added sequentially to the mixture during the mixing process.

In some embodiments, the selection of the pharmaceutically acceptable excipients and the method of blending are adapted in order to overcome any mixing, flow and fill issues or punch issues with the composition. In some embodiments, the composition comprising a substituted or unsubstituted diindolylmethane is provided in micro-encapsulated form, such that the powder particles have a tendency to clump together. In some embodiments, the composition comprising a first component, comprising a substituted or unsubstituted diindolylmethane is blended or mixed using a method that is adapted to avoid creating hot spots of increased concentrations of the active ingredients.

### NON-LIMITING EMBODIMENTS

The following specific, non-limiting embodiments are to be construed as merely illustrative, and do not limit the present disclosure of the scope of the disclosure. Without further elaboration, it is believed that one skilled in the art can, based on the description herein, utilize the present disclosure to its fullest extent.
1. A topical composition for treating a skin disease or condition in a subject, wherein the composition comprises emulsified substituted or unsubstituted diindolylmethane and a pharmaceutically acceptable carrier, and wherein the composition provides therapeutically effective amount of the substituted or unsubstituted diindolylmethane in the subject's skin affected by the skin disease or condition.
2. A topical composition for treating a skin disease or condition in a subject, wherein the composition comprises from about 0.1 wt % to about 10 wt % of substituted or unsubstituted diindolylmethane and a pharmaceutically acceptable carrier, and the composition provides therapeutically effective amount of the substituted or unsubstituted diindolylmethane in the subject's skin affected by the skin disease or condition.
3. A topical composition for treating a skin disease or condition in a subject, wherein the composition comprises from about 0.1 wt % to about 10 wt % of emulsified substituted or unsubstituted diindolylmethane and a pharmaceutically acceptable carrier, and the composition provides therapeutically effective amount of the substituted or unsubstituted diindolylmethane in the subject's skin affected by the skin disease or condition.
4. The topical composition of any one of embodiments 1-3, wherein the administration by topical route increases the dermal bioavailability of the substituted or unsubstituted diindolylmethane by about 1-fold to about 1000-fold compared to the dermal bioavailability of the substituted or unsubstituted diindolylmethane of a comparative composition administered by oral route at the same delivery dose.
5. The topical composition of any one of embodiments 1-3, wherein the administration by topical route increases the dermal bioavailability of the substituted or unsubstituted diindolylmethane by about 1-fold compared to the dermal bioavailability of the substituted or unsubstituted diindolylmethane of a comparative composition administered by oral route at the same delivery dose.
6. The topical composition of any one of embodiments 1-3, wherein the administration by topical route increases the dermal bioavailability of the substituted or unsubstituted diindolylmethane by about 2-fold compared to the dermal bioavailability of the substituted or unsubstituted diindolylmethane of a comparative composition administered by oral route at the same delivery dose.
7. The topical composition of any one of embodiments 1-3, wherein the administration by topical route increases the dermal bioavailability of the substituted or unsubstituted diindolylmethane by about 3-fold compared to the dermal bioavailability of the substituted or unsubstituted diindolylmethane of a comparative composition administered by oral route at the same delivery dose.
8. The topical composition of any one of embodiment 1-3, wherein the administration by topical route increases the dermal bioavailability of the substituted or unsubstituted diindolylmethane by about 4-fold compared to the dermal bioavailability of the substituted or unsubstituted diindolylmethane of a comparative composition administered by oral route at the same delivery dose.
9. The topical composition of any one of embodiments 1-3, wherein the administration by topical route increases the dermal bioavailability of the substituted or unsubstituted diindolylmethane by about 5-fold compared to the dermal bioavailability of the substituted or unsubstituted diindolylmethane of a comparative composition administered by oral route at the same delivery dose.
10. The topical composition of any one of embodiments 1-3, wherein the administration by topical route increases the dermal bioavailability of the substituted or unsubstituted diindolylmethane by about 10-fold compared to the dermal bioavailability of the substituted or unsubstituted diindolylmethane of a comparative composition administered by oral route at the same delivery dose.
11. The topical composition of any one of embodiments 1-3, wherein the administration by topical route increases the dermal bioavailability of the substituted or unsubstituted diindolylmethane by about 20-fold compared to the dermal bioavailability of the substituted or unsubstituted diindolylmethane of a comparative composition administered by oral route at the same delivery dose.
12. The topical composition of any one of embodiments 1-3, wherein the administration by topical route increases the dermal bioavailability of the substituted or unsubstituted diindolylmethane by about 30-fold compared to the dermal bioavailability of the substituted or unsubstituted diindolylmethane of a comparative composition administered by oral route at the same delivery dose.
13. The topical composition of any one of embodiments 1-3, wherein the administration by topical route increases the dermal bioavailability of the substituted or unsubstituted diindolylmethane by about 40-fold compared to the dermal bioavailability of the substituted or unsubstituted diindolylmethane of a comparative composition administered by oral route at the same delivery dose.
14. The topical composition of any one of embodiments 1-3, wherein the administration by topical route increases the dermal bioavailability of the substituted or unsubstituted diindolylmethane by about 50-fold compared to the dermal bioavailability of the substituted or unsubstituted diindolylmethane of a comparative composition administered by oral route at the same delivery dose.
15. The topical composition of any one of embodiments 1-3, wherein the administration by topical route increases the dermal bioavailability of the substituted or unsubstituted diindolylmethane by about 60-fold compared to the dermal bioavailability of the substituted or unsubstituted diindolylmethane of a comparative composition administered by oral route at the same delivery dose.
16. The topical composition of any one of embodiments 1-3, wherein the administration by topical route increases the dermal bioavailability of the substituted or unsubstituted diindolylmethane by about 70-fold compared to the dermal bioavailability of the substituted or unsubstituted diindolylmethane of a comparative composition administered by oral route at the same delivery dose.
17. The topical composition of any one of embodiments 1-3, wherein the administration by topical route increases the dermal bioavailability of the substituted or unsubstituted diindolylmethane by about 80-fold compared to the dermal bioavailability of the substituted or unsubstituted diindolylmethane of a comparative composition administered by oral route at the same delivery dose.
18. The topical composition of any one of embodiments 1-3, wherein the administration by topical route increases the dermal bioavailability of the substituted or unsubstituted diindolylmethane by about 100-fold compared to the dermal bioavailability of the substituted or unsubstituted diindolylmethane of a comparative composition administered by oral route at the same delivery dose.
19. The topical composition of any one of embodiments 1-3, wherein the administration by topical route increases the dermal bioavailability of the substituted or unsubstituted diindolylmethane by about 200-fold compared to the dermal bioavailability of the substituted or unsubstituted diindolylmethane of a comparative composition administered by oral route at the same delivery dose.
20. The topical composition of any one of embodiments 1-3, wherein the administration by topical route increases the dermal bioavailability of the substituted or unsubstituted diindolylmethane by about 300-fold compared to the dermal bioavailability of the substituted or unsubstituted diindolylmethane of a comparative composition administered by oral route at the same delivery dose.
21. The topical composition of any one of embodiments 1-3, wherein the administration by topical route increases the dermal bioavailability of the substituted or unsubstituted diindolylmethane by about 400-fold compared to the dermal bioavailability of the substituted or unsubstituted diindolylmethane of a comparative composition administered by oral route at the same delivery dose.
22. The topical composition of any one of embodiments 1-3, wherein the administration by topical route increases the dermal bioavailability of the substituted or unsubstituted diindolylmethane by about 500-fold compared to the dermal bioavailability of the substituted or unsubstituted diindolylmethane of a comparative composition administered by oral route at the same delivery dose.
23. The topical composition of any one of embodiments 1-3, wherein the administration by topical route increases the dermal bioavailability of the substituted or unsubstituted diindolylmethane by about 600-fold compared to the dermal bioavailability of the substituted or unsubstituted diindolylmethane of a comparative composition administered by oral route at the same delivery dose.
24. The topical composition of any one of embodiments 1-3, wherein the administration by topical route increases the dermal bioavailability of the substituted or unsubstituted diindolylmethane by about 700-fold compared to the dermal bioavailability of the substituted or unsubstituted diindolylmethane of a comparative composition administered by oral route at the same delivery dose.
25. The topical composition of any one of embodiments 1-3, wherein the administration by topical route increases the dermal bioavailability of the substituted or unsubstituted diindolylmethane by about 800-fold compared to the dermal bioavailability of the substituted or unsubstituted diindolylmethane of a comparative composition administered by oral route at the same delivery dose.
26. The topical composition of any one of embodiments 1-3, wherein the administration by topical route increases the dermal bioavailability of the substituted or unsubstituted diindolylmethane by about 900-fold compared to the dermal bioavailability of the substituted or unsubstituted diindolylmethane of a comparative composition administered by oral route at the same delivery dose.
27. The topical composition of any one of embodiments 1-3, wherein the administration by topical route increases the dermal bioavailability of the substituted or unsubstituted diindolylmethane by about 1000-fold compared to the dermal bioavailability of the substituted or unsubstituted diindolylmethane of a comparative composition administered by oral route at the same delivery dose.
28. The topical composition of any one of embodiments 4-27, wherein the dermal bioavailability of the substituted or unsubstituted diindolylmethane is measured following administration of a dose comprising less than 15 mg, preferably less than 10 mg, more preferably less than 5 mg of the substituted or unsubstituted diindolylmethane.
29. The topical composition of any one of embodiments 1-28, wherein following administration of the composition by topical route the Cmax of the substituted or unsubstituted diindolylmethane in skin increases by about 1-fold to about 1000-fold compared to the Cmax following administration of a comparative composition by oral route at the same delivery dose.
30. The topical composition of any one of embodiments 1-28, wherein following administration of the composition by topical route the Cmax of the substituted or unsubstituted diindolylmethane in skin increases by about 2-fold compared to the Cmax following administration of a comparative composition by oral route at the same delivery dose.
31. The topical composition of any one of embodiments 1-28, wherein following administration of the composition by topical route the Cmax of the substituted or unsubstituted diindolylmethane in skin increases by about 3-fold compared to the Cmax following administration of a comparative composition by oral route at the same delivery dose.
32. The topical composition of any one of embodiments 1-28, wherein following administration of the composition by topical route the Cmax of the substituted or unsubstituted diindolylmethane in skin increases by about 4-fold compared to the Cmax following administration of a comparative composition by oral route at the same delivery dose.
33. The topical composition of any one of embodiments 1-28, wherein following administration of the composition by topical route the Cmax of the substituted or unsubstituted diindolylmethane in skin increases by about 5-fold compared to the Cmax following administration of a comparative composition by oral route at the same delivery dose.
34. The topical composition of any one of embodiments 1-28, wherein following administration of the composition by topical route the Cmax of the substituted or unsubstituted diindolylmethane in skin increases by about 10-fold compared to the Cmax following administration of a comparative composition by oral route at the same delivery dose.
35. The topical composition of any one of embodiments 1-28, wherein following administration of the composition by topical route the Cmax of the substituted or unsubstituted diindolylmethane in skin increases by about 20-fold compared to the Cmax following administration of a comparative composition by oral route at the same delivery dose.
36. The topical composition of any one of embodiments 1-28, wherein following administration of the composition by topical route the Cmax of the substituted or unsubstituted diindolylmethane in skin increases by about 30-fold compared to the Cmax following administration of a comparative composition by oral route at the same delivery dose.
37. The topical composition of any one of embodiments 1-28, wherein following administration of the composition by topical route the Cmax of the substituted or unsubstituted diindolylmethane in skin increases by about 40-fold compared to the Cmax following administration of a comparative composition by oral route at the same delivery dose.
38. The topical composition of any one of embodiments 1-28, wherein following administration of the composition by topical route the Cmax of the substituted or unsubstituted diindolylmethane in skin increases by about 50-fold compared to the Cmax following administration of a comparative composition by oral route at the same delivery dose.
39. The topical composition of any one of embodiments 1-28, wherein following administration of the composition by topical route the Cmax of the substituted or unsubstituted diindolylmethane in skin increases by about 60-fold compared to the Cmax following administration of a comparative composition by oral route at the same delivery dose.
40. The topical composition of any one of embodiments 1-28, wherein following administration of the composition by topical route the Cmax of the substituted or unsubstituted diindolylmethane in skin increases by about 70-fold compared to the Cmax following administration of a comparative composition by oral route at the same delivery dose.
41. The topical composition of any one of embodiments 1-28, wherein following administration of the composition by topical route the Cmax of the substituted or unsubstituted diindolylmethane in skin increases by about 80-fold compared to the Cmax following administration of a comparative composition by oral route at the same delivery dose.
42. The topical composition of any one of embodiments 1-28, wherein following administration of the composition by topical route the Cmax of the substituted or unsubstituted diindolylmethane in skin increases by about 90-fold compared to the Cmax following administration of a comparative composition by oral route at the same delivery dose.
43. The topical composition of any one of embodiments 1-28, wherein following administration of the composition by topical route the Cmax of the substituted or unsubstituted diindolylmethane in skin increases by about 100-fold compared to the Cmax following administration of a comparative composition by oral route at the same delivery dose.
44. The topical composition of any one of embodiments 1-28, wherein following administration of the composition by topical route the Cmax of the substituted or unsubstituted diindolylmethane in skin increases by about 200-fold compared to the Cmax following administration of a comparative composition by oral route at the same delivery dose.
45. The topical composition of any one of embodiments 1-28, wherein following administration of the composition by topical route the Cmax of the substituted or unsubstituted diindolylmethane in skin increases by about 300-fold compared to the Cmax following administration of a comparative composition by oral route at the same delivery dose.
46. The topical composition of any one of embodiments 1-28, wherein following administration of the composition by topical route the Cmax of the substituted or unsubstituted diindolylmethane in skin increases by about 400-fold compared to the Cmax following administration of a comparative composition by oral route at the same delivery dose.
47. The topical composition of any one of embodiments 1-28, wherein following administration of the composition by topical route the Cmax of the substituted or unsubstituted diindolylmethane in skin increases by about 500-fold compared to the Cmax following administration of a comparative composition by oral route at the same delivery dose.
48. The topical composition of any one of embodiments 1-28, wherein following administration of the composition by topical route the Cmax of the substituted or unsubstituted diindolylmethane in skin increases by about 600-fold compared to the Cmax following administration of a comparative composition by oral route at the same delivery dose.
49. The topical composition of any one of embodiments 1-28, wherein following administration of the composition by topical route the Cmax of the substituted or unsubstituted diindolylmethane in skin increases by about 700-fold compared to the Cmax following administration of a comparative composition by oral route at the same delivery dose.
50. The topical composition of any one of embodiments 1-28, wherein following administration of the composition by topical route the Cmax of the substituted or unsubstituted diindolylmethane in skin increases by about 800-fold compared to the Cmax following administration of a comparative composition by oral route at the same delivery dose.
51. The topical composition of any one of embodiments 1-28, wherein following administration of the composition by topical route the Cmax of the substituted or unsubstituted diindolylmethane in skin increases by about 900-fold compared to the Cmax following administration of a comparative composition by oral route at the same delivery dose.
52. The topical composition of any one of embodiments 1-28, wherein following administration of the composition by topical route the Cmax of the substituted or unsubstituted diindolylmethane in skin increases by about 1000-fold compared to the Cmax following administration of a comparative composition by oral route at the same delivery dose.
53. The topical composition of any one of embodiments 29-52, wherein the Cmax of the substituted or unsubstituted diindolylmethane in skin is measured following administration of a dose comprising less than 15 mg, preferably less than 10 mg, more preferably less than 5 mg of the substituted or unsubstituted diindolylmethane.
54. The topical composition of any one of embodiments 1-53, wherein following administration of the composition by topical route the AUC of the substituted or unsubstituted diindolylmethane in skin increases by about 1-fold to about 1000-fold compared to the AUC following administration of a comparative composition by oral route at the same delivery dose.
55. The topical composition of any one of embodiments 1-53, wherein following administration of the composition by topical route the AUC of the substituted or unsubstituted diindolylmethane in skin increases by about 2-fold compared to the AUC following administration of a comparative composition by oral route at the same delivery dose.
56. The topical composition of any one of embodiments 1-53, wherein following administration of the composition by topical route the AUC of the substituted or unsubstituted diindolylmethane in skin increases by about 3-fold compared to the AUC following administration of a comparative composition by oral route at the same delivery dose.
57. The topical composition of any one of embodiments 1-53, wherein following administration of the composition by topical route the AUC of the substituted or unsubstituted diindolylmethane in skin increases by about 4-fold compared to the AUC following administration of a comparative composition by oral route at the same delivery dose.
58. The topical composition of any one of embodiments 1-53, wherein following administration of the composition by topical route the AUC of the substituted or unsubstituted diindolylmethane in skin increases by about 5-fold compared to the AUC following administration of a comparative composition by oral route at the same delivery dose.
59. The topical composition of any one of embodiments 1-53, wherein following administration of the composition by topical route the AUC of the substituted or unsubstituted diindolylmethane in skin increases by about 6-fold compared to the AUC following administration of a comparative composition by oral route at the same delivery dose.
60. The topical composition of any one of embodiments 1-53, wherein following administration of the composition by topical route the AUC of the substituted or unsubstituted diindolylmethane in skin increases by about 7-fold compared to the AUC following administration of a comparative composition by oral route at the same delivery dose.
61. The topical composition of any one of embodiments 1-53, wherein following administration of the composition by topical route the AUC of the substituted or unsubstituted diindolylmethane in skin increases by about 8-fold compared to the AUC following administration of a comparative composition by oral route at the same delivery dose.
62. The topical composition of any one of embodiments 1-53, wherein following administration of the composition by topical route the AUC of the substituted or unsubstituted diindolylmethane in skin increases by about 9-fold compared to the AUC following administration of a comparative composition by oral route at the same delivery dose.
63. The topical composition of any one of embodiments 1-53, wherein following administration of the composition by topical route the AUC of the substituted or unsubstituted diindolylmethane in skin increases by about 10-fold compared to the AUC following administration of a comparative composition by oral route at the same delivery dose.
64. The topical composition of any one of embodiments 1-53, wherein following administration of the composition by topical route the AUC of the substituted or unsubstituted diindolylmethane in skin increases by about 20-fold compared to the AUC following administration of a comparative composition by oral route at the same delivery dose.
65. The topical composition of any one of embodiments 1-53, wherein following administration of the composition by topical route the AUC of the substituted or unsubstituted diindolylmethane in skin increases by about 30-fold compared to the AUC following administration of a comparative composition by oral route at the same delivery dose.
66. The topical composition of any one of embodiments 1-53, wherein following administration of the composition by topical route the AUC of the substituted or unsubstituted diindolylmethane in skin increases by about 40-fold compared to the AUC following administration of a comparative composition by oral route at the same delivery dose.
67. The topical composition of any one of embodiments 1-53, wherein following administration of the composition by topical route the AUC of the substituted or unsubstituted diindolylmethane in skin increases by about 50-fold compared to the AUC following administration of a comparative composition by oral route at the same delivery dose.
68. The topical composition of any one of embodiments 1-53, wherein following administration of the composition by topical route the AUC of the substituted or unsubstituted diindolylmethane in skin increases by about 60-fold compared to the AUC following administration of a comparative composition by oral route at the same delivery dose.
69. The topical composition of any one of embodiments 1-53, wherein following administration of the composition by topical route the AUC of the substituted or unsubstituted diindolylmethane in skin increases by about 70-fold compared to the AUC following administration of a comparative composition by oral route at the same delivery dose.
70. The topical composition of any one of embodiments 1-53, wherein following administration of the composition by topical route the AUC of the substituted or unsubstituted diindolylmethane in skin increases by about 80-fold compared to the AUC following administration of a comparative composition by oral route at the same delivery dose.
71. The topical composition of any one of embodiments 1-53, wherein following administration of the composition by topical route the AUC of the substituted or unsubstituted diindolylmethane in skin increases by about 90-fold compared to the AUC following administration of a comparative composition by oral route at the same delivery dose.
72. The topical composition of any one of embodiments 1-53, wherein following administration of the composition by topical route the AUC of the substituted or unsubstituted diindolylmethane in skin increases by about 100-fold compared to the AUC following administration of a comparative composition by oral route at the same delivery dose.
73. The topical composition of any one of embodiments 1-53, wherein following administration of the composition by topical route the AUC of the substituted or unsubstituted diindolylmethane in skin increases by about 200-fold compared to the AUC following administration of a comparative composition by oral route at the same delivery dose.
74. The topical composition of any one of embodiments 1-53, wherein following administration of the composition by topical route the AUC of the substituted or unsubstituted diindolylmethane in skin increases by about 300-fold compared to the AUC following administration of a comparative composition by oral route at the same delivery dose.
75. The topical composition of any one of embodiments 1-53, wherein following administration of the composition by topical route the AUC of the substituted or unsubstituted diindolylmethane in skin increases by about 400-fold compared to the AUC following administration of a comparative composition by oral route at the same delivery dose.
76. The topical composition of any one of embodiments 1-53, wherein following administration of the composition by topical route the AUC of the substituted or unsubstituted diindolylmethane in skin increases by about 500-fold compared to the AUC following administration of a comparative composition by oral route at the same delivery dose.
77. The topical composition of any one of embodiments 1-53, wherein following administration of the composition by topical route the AUC of the substituted or unsubstituted diindolylmethane in skin increases by about 600-fold compared to the AUC following administration of a comparative composition by oral route at the same delivery dose.
78. The topical composition of any one of embodiments 1-53, wherein following administration of the composition by topical route the AUC of the substituted or unsubstituted diindolylmethane in skin increases by about 700-fold compared to the AUC following administration of a comparative composition by oral route at the same delivery dose.
79. The topical composition of any one of embodiments 1-53, wherein following administration of the composition by topical route the AUC of the substituted or unsubstituted diindolylmethane in skin increases by about 800-fold compared to the AUC following administration of a comparative composition by oral route at the same delivery dose.
80. The topical composition of any one of embodiments 1-53, wherein following administration of the composition by topical route the AUC of the substituted or unsubstituted diindolylmethane in skin increases by about 900-fold compared to the AUC following administration of a comparative composition by oral route at the same delivery dose.
81. The topical composition of any one of embodiments 1-53, wherein following administration of the composition by topical route the AUC of the substituted or unsubstituted diindolylmethane in skin increases by about 1000-fold compared to the AUC following administration of a comparative composition by oral route at the same delivery dose.
82. The topical composition of any one of embodiments 54-81, wherein the AUC of the substituted or unsubstituted diindolylmethane in skin is measured following administration of a dose comprising less than 15 mg, preferably less than 10 mg, more preferably less than 5 mg of the substituted or unsubstituted diindolylmethane.
83. The topical composition of any one of embodiments 1-82, wherein following administration of the composition by topical route the Tmax of the substituted or unsubstituted diindolylmethane in skin decreases by about 1-fold to about 1000-fold compared to the Tmax following administration of a comparative composition by oral route at the same delivery dose.
84. The topical composition of any one of embodiments 1-82, wherein following administration of the composition by topical route the Tmax of the substituted or unsubstituted diindolylmethane in skin decreases by about 1-fold compared to the Tmax following administration of a comparative composition by oral route at the same delivery dose.
85. The topical composition of any one of embodiments 1-82, wherein following administration of the composition by topical route the Tmax of the substituted or unsubstituted diindolylmethane in skin decreases by about 2-fold compared to the Tmax following administration of a comparative composition by oral route at the same delivery dose.
86. The topical composition of any one of embodiments 1-82, wherein following administration of the composition by topical route the Tmax of the substituted or unsubstituted diindolylmethane in skin decreases by about 3-fold compared to the Tmax following administration of a comparative composition by oral route at the same delivery dose.
87. The topical composition of any one of embodiments 1-82, wherein following administration of the composition by topical route the Tmax of the substituted or unsubstituted diindolylmethane in skin decreases by about 4-fold compared to the Tmax following administration of a comparative composition by oral route at the same delivery dose.
88. The topical composition of any one of embodiments 1-82, wherein following administration of the composition by topical route the Tmax of the substituted or unsubstituted diindolylmethane in skin decreases by about 5-fold compared to the Tmax following administration of a comparative composition by oral route at the same delivery dose.
89. The topical composition of any one of embodiments 1-82, wherein following administration of the composition by topical route the Tmax of the substituted or unsubstituted diindolylmethane in skin decreases by about 10-fold compared to the Tmax following administration of a comparative composition by oral route at the same delivery dose.
90. The topical composition of any one of embodiments 1-82, wherein following administration of the composition by topical route the Tmax of the substituted or unsubstituted diindolylmethane in skin decreases by about 20-fold compared to the Tmax following administration of a comparative composition by oral route at the same delivery dose.
91. The topical composition of any one of embodiments 1-82, wherein following administration of the composition by topical route the Tmax of the substituted or unsubstituted diindolylmethane in skin decreases by about 30-fold compared to the Tmax following administration of a comparative composition by oral route at the same delivery dose.
92. The topical composition of any one of embodiments 1-82, wherein following administration of the composition by topical route the Tmax of the substituted or unsubstituted diindolylmethane in skin decreases by about 40-fold compared to the Tmax following administration of a comparative composition by oral route at the same delivery dose.
93. The topical composition of any one of embodiments 1-82, wherein following administration of the composition by topical route the Tmax of the substituted or unsubstituted diindolylmethane in skin decreases by about 50-fold compared to the Tmax following administration of a comparative composition by oral route at the same delivery dose.
94. The topical composition of any one of embodiments 1-82, wherein following administration of the composition by topical route the Tmax of the substituted or unsubstituted diindolylmethane in skin decreases by about 60-fold compared to the Tmax following administration of a comparative composition by oral route at the same delivery dose.
95. The topical composition of any one of embodiments 1-82, wherein following administration of the composition by topical route the Tmax of the substituted or unsubstituted diindolylmethane in skin decreases by about 70-fold compared to the Tmax following administration of a comparative composition by oral route at the same delivery dose.
96. The topical composition of any one of embodiments 1-82, wherein following administration of the composition by topical route the Tmax of the substituted or unsubstituted diindolylmethane in skin decreases by about 80-fold compared to the Tmax following administration of a comparative composition by oral route at the same delivery dose.
97. The topical composition of any one of embodiments 1-82, wherein following administration of the composition by topical route the Tmax of the substituted or unsubstituted diindolylmethane in skin decreases by about 90-fold compared to the Tmax following administration of a comparative composition by oral route at the same delivery dose.
98. The topical composition of any one of embodiments 1-82, wherein following administration of the composition by topical route the Tmax of the substituted or unsubstituted diindolylmethane in skin decreases by about 100-fold compared to the Tmax following administration of a comparative composition by oral route at the same delivery dose.
99. The topical composition of any one of embodiments 1-82, wherein following administration of the composition by topical route the Tmax of the substituted or unsubstituted diindolylmethane in skin decreases by about 200-fold compared to the Tmax following administration of a comparative composition by oral route at the same delivery dose.
100. The topical composition of any one of embodiments 1-82, wherein following administration of the composition by topical route the Tmax of the substituted or unsubstituted diindolylmethane in skin decreases by about 300-fold compared to the Tmax following administration of a comparative composition by oral route at the same delivery dose.
101. The topical composition of any one of embodiments 1-82, wherein following administration of the composition by topical route the Tmax of the substituted or unsubstituted diindolylmethane in skin decreases by about 400-fold compared to the Tmax following administration of a comparative composition by oral route at the same delivery dose.
102. The topical composition of any one of embodiments 1-82, wherein following administration of the composition by topical route the Tmax of the substituted or unsubstituted diindolylmethane in skin decreases by about 500-fold compared to the Tmax following administration of a comparative composition by oral route at the same delivery dose.
103. The topical composition of any one of embodiments 1-82, wherein following administration of the composition by topical route the Tmax of the substituted or unsubstituted diindolylmethane in skin decreases by about 600-fold compared to the Tmax following administration of a comparative composition by oral route at the same delivery dose.
104. The topical composition of any one of embodiments 1-82, wherein following administration of the composition by topical route the Tmax of the substituted or unsubstituted diindolylmethane in skin decreases by about 700-fold compared to the Tmax following administration of a comparative composition by oral route at the same delivery dose.
105. The topical composition of any one of embodiments 1-82, wherein following administration of the composition by topical route the Tmax of the substituted or unsubstituted diindolylmethane in skin decreases by about 800-fold compared to the Tmax following administration of a comparative composition by oral route at the same delivery dose.
106. The topical composition of any one of embodiments 1-82, wherein following administration of the composition by topical route the Tmax of the substituted or unsubstituted diindolylmethane in skin decreases by about 900-fold compared to the Tmax following administration of a comparative composition by oral route at the same delivery dose.
107. The topical composition of any one of embodiments 1-82, wherein following administration of the composition by topical route the Tmax of the substituted or unsubstituted diindolylmethane in skin decreases by about 1000-fold compared to the Tmax following administration of a comparative composition by oral route at the same delivery dose.
108. The topical composition of any one of embodiments 83-107, wherein the Tmax of the substituted or unsubstituted diindolylmethane in skin is measured following administration of a dose comprising less than 15 mg, preferably less than 10 mg, more preferably less than 5 mg of the substituted or unsubstituted diindolylmethane.
109. The topical composition of any one of embodiments 1-108, wherein the composition comprises from about 0.1 wt % to about 8.0 wt % of substituted or unsubstituted diindolylmethane.
110. The topical composition of any one of embodiments 1-108, wherein the composition comprises from about 0.1 wt % to about 5.0 wt % of substituted or unsubstituted diindolylmethane.
111. The topical composition of any one of embodiments 1-108, wherein the composition comprises from about 0.1 wt % to about 4.0 wt % of substituted or unsubstituted diindolylmethane.
112. The topical composition of any one of embodiments 1-108, wherein the composition comprises from about 0.1 wt % to about 3.0 wt % of substituted or unsubstituted diindolylmethane.
113. The topical composition of any one of embodiments 1-108, wherein the composition comprises from about 0.2 wt % to about 3.0 wt % of substituted or unsubstituted diindolylmethane.
114. The topical composition of any one of embodiments 1-108, wherein the composition comprises from about 0.3 wt % to about 3.0 wt % of substituted or unsubstituted diindolylmethane.
115. The topical composition of any one of embodiments 1-108, wherein the composition comprises from about 0.5 wt % to about 2.5 wt % of substituted or unsubstituted diindolylmethane.
116. The topical composition of any one of embodiments 1-108, wherein the composition comprises from about 0.3 wt % to about 0.8 wt % of substituted or unsubstituted diindolylmethane.
117. The topical composition of any one of embodiments 1-108, wherein the composition comprises about 0.5 wt % of substituted or unsubstituted diindolylmethane.
118. The topical composition of any one of embodiments 1-108, wherein the composition comprises from about 0.8 wt % to about 1.2 wt % of substituted or unsubstituted diindolylmethane.
119. The topical composition of any one of embodiments 1-108, wherein the composition comprises from about 1.0 wt % of substituted or unsubstituted diindolylmethane.
120. The topical composition of any one of embodiments 1-108, wherein the composition comprises from about 1.2 wt % to about 2.0 wt % of substituted or unsubstituted diindolylmethane.
121. The topical composition of any one of embodiments 1-108, wherein the composition comprises about 1.5 wt % of substituted or unsubstituted diindolylmethane.
122. The topical composition of any one of embodiments 1-108, wherein the composition comprises from about 2.0 wt % to about 3.0 wt % of substituted or unsubstituted diindolylmethane.
123. The topical composition of any one of embodiments 1-108, wherein the composition comprises about 2.5 wt % of substituted or unsubstituted diindolylmethane.
124. The topical composition of any one of embodiments 1-123, wherein the substituted or unsubstituted diindolylmethane is unsubstituted diindolylmethane.
125. The topical composition of any one of embodiments 1-123, wherein the substituted or unsubstituted diindolylmethane is unsubstituted 3,3′-diindolylmethane.
126. The topical composition of any one of embodiments 1-125, wherein the composition further comprises a retinoid compound.
127. The topical composition of embodiment 126, wherein the retinoid compound is a substituted or unsubstituted retinoic acid.
128. The topical composition of embodiment 126, wherein the retinoid compound is vitamin A.
129. The topical composition of embodiment 126, wherein the retinoid compound is retinyl palmitate.
130. The topical composition of any one of embodiments 1-129, wherein the composition is essentially free of any retinoid compound.
131. The topical composition of any one of embodiments 1-129, wherein the composition is free of any retinoid compound.
132. The topical composition of any one of embodiments 1-129, wherein the composition is essentially free of any therapeutically active agent other than the substituted or unsubstituted diindolylmethane.
133. The topical composition of any one of embodiments 1-129, wherein the composition is free of any therapeutically active agent other than the substituted or unsubstituted diindolylmethane.

### Non-Limiting Examples

The following specific, non-limiting examples are to be construed as merely illustrative, and do not limit the present disclosure of the scope of the disclosure. Without further elaboration, it is believed that one skilled in the art can, based on the description herein, utilize the present disclosure to its fullest extent.

### Example 1: Topical DIM Formulation—Emulsion Gel (Emugel)

In this non-limiting example, the topical DIM composition is formulated as an emulsion gel, in which the oil phase contains the DIM compounds and the aqueous phase comprises a hydrogel. Below is a list of the ingredients for the emulsion gel formulation.
Water—aqueous carrier
Glycerin—carrier
DIM—active agent (0.1-10 wt %)
Dicaprylyl carbonate ester—emollient
Ammonium acryloyldimethyltaurate/VP copolymer—rheology modifier
Carbomer—rheology modifier
Polyglyceryl—10 laurate—emulsifier
Levulinic acid—skin conditioner and preservative
Sodium anisate—preservative
Sodium levulinate—skin conditioner and preservative
Sodium hydroxide—pH adjusting agent
Tocopherol—moisturizer/antioxidant

To make the formulation, various ingredients were mixed together until a uniform emulsion gel is obtained.

### Example 2: Topical DIM Formulation-Cream

In this non-limiting example, the topical DIM composition is formulated as an emulsion cream, in which the oil phase contains the DIM compounds and the aqueous phase comprises a base cream. Below is a list of the ingredients for the emulsion cream formulation.
Water—aqueous carrier
Prunus amygdalus dulcis (sweet almond) oil
Dicaprylyl carbonate ester—emollient
Glycerin—carrier
Stearyl alcohol fatty alcohol
Polyglyceryl-3 stearate—emulsifier
Potassium cetyl phosphate—emulsifier
Glyceryl stearate citrate—emulsifier
Xanthan gum—rheology modifier/gum
Levulinic acid—skin conditioner and preservative
Ammonium acryloyldimethyltaurate/VP copolymer rheology modifier—rheology modifier
Sodium anisate—preservative
Sodium levulinate—skin conditioner and preservative
Helianthus annuus (sunflower) seed oil
Sodium hydroxide—pH adjusting agent
Rosmarinus officinalis (rosemary) leaf extract
Tocopherol—moisturizer/antioxidant

To make the formulation, various ingredients were mixed together until a uniform emulsion cream is obtained.

### Example 3: Dermal Pharmacokinetic Evaluation of Topical Formulations

Skin is the outermost largest organ in the body. It protects the body from physical, chemical, and microbial assaults and also prevents water loss from the body. The skin comprises three layers, i.e. epidermis, dermis, and subcutaneous tissue. Epidermis is made up of five layers composed of stratum corneum, stratum lucidum, stratum granulosum, stratum spinosum, and stratum basale. The stratum corneum is the outermost layer and comprise dead keratinocytes, and lamellar granules. The corneocytes and lipids form tight junctions in the stratum corneum like brick and mortar. Stratum corneum acts as a permeation and diffusional barrier for topically applied dosage form for skin disorders and transdermal drug delivery. The topical formulations are considered as a mainstay for the treatment of these skin disorders. Topical drug delivery systems are mostly preferred to deliver the drugs to the target site with minimal systemic adverse effects.

The bioavailability of the topical and transdermal administered drugs is affected by various factors. They include permeation through the skin (rate-limiting membrane stratum corneum), physicochemical properties of the drug (pKa, log P, solubility, molecular weight), excipients used in the formulation, type of formulation, presence of enzymes in skin structures, application site, and type of the skin. After the topical or transdermal application of the formulation onto the skin, the drug must permeate deeper layers by crossing various layers. Various permeability enhancement techniques are employed to increase drug transport into the stratum corneum. Consequently, skin cannot be deemed as a single compartment. The estimation of drug retained in skin layers is critical in topical and transdermal drug delivery. Therefore, there is a need to separate the skin into layers to estimate the drug permeation in different layers. Dermato-pharmacokinetic studies can be very useful for evaluation of topical product's performance and assessment in drug permeation-retention in the skin layers.

Many techniques may be used to assess dermato-pharmacokinetics. Parameters such as Cmax, Tmax, and AUC are extensively assessed in dermato-pharmacokinetic studies. Assessment of dermato-pharmacokinetics of administered topical formulations is desirable in appraising the products' safety and efficacy. Several techniques have been explored for the estimation of the drug in the different skin layers. In diseased skin, the stratum corneum barrier function is impaired, which does not hinder drug permeation. For dermato-pharmacokinetic studies, healthy skin may be more recommended than diseased skin. Using two or three techniques simultaneously provides superior benefits. Dermato-pharmacokinetic evaluation can be utilized for quality control tool for complex formulations and can provide the road map for newer product development.

### Dermato-Pharmacokinetic Evaluation Techniques

The dermato-pharmacokinetic method is employed to estimate drug concentrations in the skin layers. Although the stratum corneum might not be the site of action, it is the rate-limiting membrane for drug permeation. The dermato-pharmacokinetics is akin to blood and urine pharmacokinetic studies. The skin kinetics depends on the drug absorption and the termination of administration. Dermato-pharmacokinetics methods assume three mechanisms: i) stratum corneum behavior as the rate-limiting membrane in drug absorption; ii) diffusion of the drug into the dermis is directly proportional to the concentration of drug at the stratum corneum; iii) The drug concentrations in the skin layers (stratum corneum, epidermis, dermis) depict the dermatological efficacy of the drug [Citation2]. The parameters in dermato-pharmacokinetics include Cmax (maximum drug concentration in the skin layers), Tmax (time required to reach Cmax), and Area under the curve (AUC) in stratum corneum, epidermis, dermis. The Cmax. Tmax and AUC parameters are used to evaluate drug molecules pharmacokinetics in the skin layers [Citation1]. The dermato-pharmacokinetic profile of the topically applied drugs is estimated using in-vitro and in-vivo techniques as discussed in the following section.

### Tape Stripping

Tape stripping is the most widely employed technique for the estimation of the drug in the stratum corneum. The utilization of tape stripping for quantification of drugs in the stratum corneum was proposed in the draft guidance by the United States Department of Health and Human Services in 1998 to assess the bioequivalence of the topical products. This method can be utilized in both in-vitro and in-vivo drug permeation studies for topical delivery systems applied to the skin. It is uncomplicated, easily implemented for quantification of drug retained in the stratum corneum. This technique is minimally invasive in the in-vivo conditions (Preclinical and clinical studies). It involves removing the stratum corneum layer one after another using adhesive tape strips. The tape strip is applied with the pressure using a roller to ensure uniform and proper adhesion onto the skin surface. The tape strip's application pressure should maintain uniform throughout the tape stripping process to get reproducible results. The collected tape strips are added to the solvent of interest in which the drug is highly soluble to extract the drug from the adhesive. The drug extracted is subjected to centrifugation and quantified using a validated analytical technique. The typical limitations with the tape stripping technique is the interference of adhesive, and the number of strips required to remove the stratum corneum is varied from person to person and site of application. There is a requirement of validating the procedure before the utilization of the process in preclinical and clinical studies. The number of stratum corneum layers differs in animals (mice, rat, rabbit, guinea pig, goat, pig) and humans.

The tape stripping technique is validated using the weighing method or the protein estimation method. In weighing method, the tape strip's weight is noted before and after tape stripping, and the protein levels of corneocytes are measured to determine the stratum corneum layers in the skin. Olesen et al. reported that protein removal was decreased with an increase in tape stripping depth. The study results revealed that tape stripping can be used to skin barrier research without discomfort and scars compared to skin biopsies.

### Microdialysis and Open-Flow Microperfusion

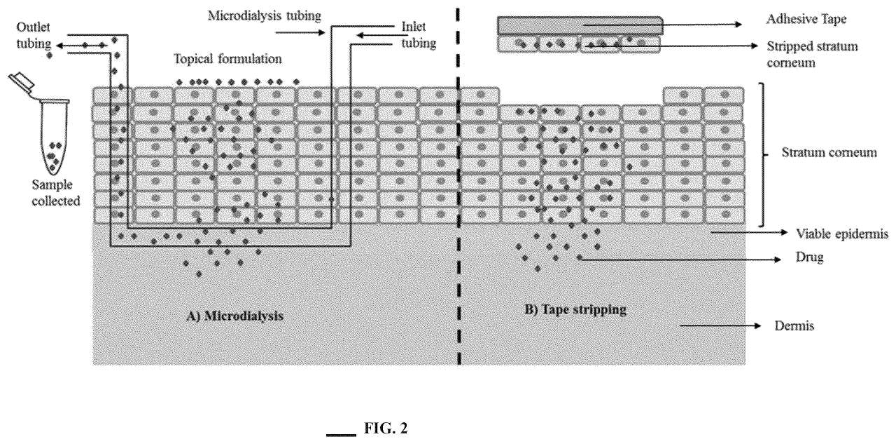

A microdialysis and open-flow microperfusion techniques are employed to assess the bioequivalence of the topical drug product. The drug concentration is determined in the dermis and hypodermis by implanting a semipermeable membrane probe guided by a needle. The probe is perfused with the sterile buffer, which mimics the blood vessels, equilibrates by interstitial fluids, and the drug molecules diffuse from tissues to the buffer. Isotonic saline/Ringer's lactate solution is perfused at the rate of 1-5 μL. FIG. 2 depicts the microdialysis and tape striping technique.

### Vasoconstrictor Assay

Vasoconstrictor assay is also termed as the skin blanching assay, which is only limited to the corticosteroid formulations bioavailability assessment. After applying corticosteroids, the local vasoconstriction can be measured by visual examination, chromametry, and analysis using digital imaging. In chromametry, the white light gets reflected into three different constraints red-green, yellow-blue, and light-dark. Using a digital image of 0.5 cm2 with 300 dots per inch is analyzed for the above constraints. Visual inspection is an alternative to chromametry; the visual responses are given in the range of 0-4. Whereas 0 indicates no blanching, and required further no blanching, whereas numbers from 1 to 4 show the increasing array of blanching (absent=0, faint=1, faint-moderate=2, moderate-strong=3, and strong-intense blanching=4) practical application.

### Microscopic and Spectroscopic Methods

Along with the above methods, microscopic and spectroscopic techniques have been widely explored for the assessment of drug distribution. In microscopy analysis techniques, the dye (fluorescein, Nile red, and 5-bromodeoxyuridine, coumarin c6) is utilized for the assessment of the distribution and penetration into deeper layers of the skin. The dye may not exhibit similar behavior as a drug, which is the major limitation of the technique. In spectroscopic techniques, Attenuated Total Reflectance (ATR) or Fourier-Transform Infrared Spectroscopy (FTIR) is employed to estimate the modifications in the stratum corneum modifications. The conformation of proteins in outer skin layers, an assortment of lipids, treatment with chemical enhancers, and employing physical methods may modify permeability. The shift in the infrared bands illustrates the relative alterations between treated and untreated skin. The combination of Raman spectroscopy and confocal microscopy attracted great attention in estimating drug distribution in skin samples. The interference of water in Raman spectroscopy is null; it works on the principle of inelastic scattering of monochromatic light from a laser beam. The chemical structure, electronic configuration, and molecular bonds of detailed compound information are obtained using Raman spectroscopy.

### Dermato-Pharmacokinetic Parameters Estimation

The one compartmental model is employed for estimation of the dermato-pharmacokinetic parameters by the below-mentioned equations:

$${C}_{skin} = \frac{{K}_{p} \cdot {C}_{max}}{( {K}_{p} - {K}_{e} )} ( {e}^{- Kpt} - {e}^{- Ket} ) \qquad ( 1 )$$

$${C}_{Skin} - Drug concentration in the skin at time t .$$

$${K}_{p} = Permeation rate constant ( epidermal / dermal ) .$$

$${C}_{max} - Maximum concentration of drug achieved in the skin layer .$$

$${K}_{e} - Skin elimination rate constant from epidermis / dermis .$$

$${T}_{max} = \frac{2.303 \times {log}^{Kp / Ke}}{( Kp - Ke )} \qquad ( 2 )$$

$${T}_{max} - Time required to achieve maximum drug concentration .$$

$${C}_{max} = \frac{{FX}_{0}}{{V}_{d}} {e}^{- {K}_{e} {T}_{max}} \qquad ( 3 )$$

$$F - Fraction bioavailable$$

$$Vd - Volume of distribution$$

$$Xo - Initial amount of drug administered$$

$${AUC}_{( O - t )} = {\sum }_{i = 0}^{i = t} \frac{( {C}_{1} + {C}_{2} ) ( {T}_{2} - {T}_{1} )}{2} \qquad ( 4 )$$

$$AUC - Area under the curve$$

$${T}_{1} and {T}_{2} - Consecutive time points$$

$${C}_{1} and {C}_{2} - Drug concentrations are {C}_{1} and {C}_{2}$$

### Example 4: Improved Efficacy of Acne Treatment by Topical Administration of DIM Emulstion Gel

The objective of this study is to determine the efficacy of a diindolylmethane (DIM) formulation in treating acne following a 4 week treatment with a topical DIM formulation or an oral DIM formulation at the same delivery dosage. The DIM topical formulation for this study is prepared according to Example 1. The DIM oral formulation for this study is prepared according to U.S. Pat. No. 9,180,117.

Participants will be grouped into (a) Group 1: DIM administered by topical route and (b) Group 2: DIM administered by oral route.

Participants of Groups 1 and 2 will be administered, orally and by the topical route respectively, on days 1 through 28, a single dose of the DIM formulation twice daily. After completion of the study skin biophysical parameters related to acne, including but not limited to skin sebum and stratum corneum hydration levels, transepidermal water loss values, pH, erythema and hair growth parameters such as total number, density and proportion of anagen hair, of the study participants will be assessed.

### Example 5: Improved Efficacy of Acne Treatment by Topical Administration of DIM Emulsion Cream

The objective of this study is to determine the efficacy of a diindolylmethane (DIM) formulation in treating acne following a 4 week treatment with a topical DIM formulation or an oral DIM formulation at the same delivery dosage. The DIM topical formulation for this study is prepared according to Example 2. The DIM oral formulation for this study is prepared according to U.S. Pat. No. 9,180,117.

Participants will be grouped into (a) Group 1: DIM administered by topical route and (b) Group 2: DIM administered by oral route.

Participants of Groups 1 and 2 will be administered, orally and by the topical route respectively, on days 1 through 28, a single dose of the DIM formulation twice daily. After completion of the study skin biophysical parameters related to acne, including but not limited to skin sebum and stratum corneum hydration levels, transepidermal water loss values, pH, erythema and hair growth parameters such as total number, density and proportion of anagen hair, of the study participants will be assessed.

### Example 6: Improved Efficacy of Atopic Dermatitis Treatment by Topical Administration of DIM Emulstion Gel

The objective of this study is to determine the efficacy of a diindolylmethane (DIM) formulation in treating atopic dermatitis following a 4 week treatment with a topical DIM formulation or an oral DIM formulation at the same delivery dosage. The DIM topical formulation for this study is prepared according to Example 1. The DIM oral formulation for this study is prepared according to U.S. Pat. No. 9,180,117.

Participants will be grouped into (a) Group 1: DIM administered by topical route and (b) Group 2: DIM administered by oral route.

Participants of Groups 1 and 2 will be administered, orally and by the topical route respectively, on days 1 through 28, a single dose of the DIM formulation twice daily. After completion of the study skin biophysical parameters related to atopic dermatitis of the study participants will be assessed.

### Example 7: Improved Efficacy of Atopic Dermatitis Treatment by Topical Administration of DIM Emulsion Cream

The objective of this study is to determine the efficacy of a diindolylmethane (DIM) formulation in treating atopic dermatitis following a 4 week treatment with a topical DIM formulation or an oral DIM formulation at the same delivery dosage. The DIM topical formulation for this study is prepared according to Example 2. The DIM oral formulation for this study is prepared according to U.S. Pat. No. 9,180,117.

Participants will be grouped into (a) Group 1: DIM administered by topical route and (b) Group 2: DIM administered by topical route

Participants of Groups 1 and 2 will be administered, orally and by the topical route respectively, on days 1 through 28, a single dose of the DIM formulation twice daily. After completion of the study skin biophysical parameters related to atopic dermatitis of the study participants will be assessed.

### Example 8: Effect of the Route of Administration on the PK Profile of DIM+Vitamin a in Skin

### Survey Objectives

### Primary Objective

To assess the effects of PS topical DIM formulation on the parameters of erythema, pigmentation, sebum production, skin elasticity, stratum corneum hydration, and trans epidermal water loss, as well as severity of acne vulgaris (groups 1 to 4) and atopic dermatitis (groups 5 to 8).

### Secondary Objective

To evaluate the cosmetic acceptability of the PS topical DIM product.

### Survey Design

### Survey Design and Duration

The survey is a randomized, double blind cosmetic performance survey. 24 healthy volunteers of all genders, between the age of 18 and 45 years will be recruited, 12 with moderate acne vulgaris and 12 with moderate atopic dermatitis. 3 participants in each group will be randomly allocated to use different % of topical cream or emugel.

The study will last 16 weeks with testing taking place on site at iiaa at 0, 8, 12 and 16 weeks. At each visit participants will complete self-assessment questionnaires. A medical practitioner will assess acne/atopic dermatitis severity. Skin function will also be assessed using non-invasive measures of skin elasticity, erythema, pigmentation, sebum production, stratum corneum hydration, transepidermal water loss and topography, as well as severity of acne vulgaris (groups 1 to 4) and atopic dermatitis (groups 5 to 8). The equipment used will be Courage+Khazaka Dual 580 Multi Probe Adapter System (MPA) and the Canfield Visia Machine. All devices are considered to be manufactured and maintained at the cosmetic industry standards and are non-invasive technologies for assessing the various parameters of skin. All equipment has extensive, well established, safe use in UK and international skin research centres.

### Equipment

### Courage+Khazaka Dual 580 Multi Probe Adapter System (MPA)

The Courage and Khazaka System will be used for the skin measurements. It consists of a basic device and probes. The probes to be used for this survey consist of the cutometer to measure elasticity, corneometer to measure hydration, tewameter to measure transepidermal water loss, sebumeter to measure sebum levels, mexameter to measure melatonin and erythema, and Visioscan VC 98 to study the skin surface. All assessments are non-invasive.

### Canfield Visia Machine

The Visia machine uses cross-polarized and UV lighting to record and measure surface and subsurface skin conditions. UV photography provides the most complete data set available for sun damage assessment and analysis, including UV fluorescence imaging to reveal porphyrins, spots, wrinkles, texture, pores, brown spots, red areas and UV spots.

### Assessments

### Participant Self-Assessment

At each visit participants will complete a visual analogue score questionnaire to assess cosmetic acceptability.

### Practitioner Assessment

At each visit, a medical practitioner will assessment the severity of acne vulgaris/atopic dermatitis.

Acne vulgaris will be assessed with:
Clinical erythema index (CEI)
Comprehensive Acne Severity Scale (CASS)
Cardiff Acne Disability Index (CADI)
Global score for inflammatory and non-inflammatory lesions
Atopic dermatitis will be assessed with:
Eczema area and severity index (EASI)
SCOring Atopic Dermatitis (SCORAD)

### Participants

### Number

24 healthy volunteers of all genders, between 18 and 45 years, will be recruited. These will fall into 8 groups of 3 volunteers each: groups 1 to 4 who have moderate facial acne vulgaris; groups 5 to 8 who have moderate atopic dermatitis. Volunteers will be screened for suitability.

### Recruitment

Potential subjects will be recruited by recruitment agencies as well as local dermatologists.

### Inclusion Criteria

Healthy adult males and females aged between 18 and 45 years

### Groups 1 to 4

Volunteers with moderate facial acne vulgaris (20 to 50 inflammatory lesions/papules on the face, excluding the nose)

### Groups 5 to 8

Volunteers with moderate atopic dermatitis

### All Groups

The participant is able to read and understand the Informed Consent Form (ICF) and understand study procedures.

The participant has signed the ICF.

Female participants who are willing to use an acceptable form of contraception (including abstinence, combined oestrogen/progesterone medication, implants or intrauterine contraceptive devices). Where hormonal contraception is employed, the participant needs to have been on stable treatment for 3 months prior to screening and that no changes will be made to this treatment throughout the study. Contraception medication used purely for acne treatment is prohibited.

Currently not using any prescribed acne medications or receiving any laser/light therapy acne treatments.

Willing to stop any current over-the-counter non-prescribed acne treatments from screening until the end of the study.

Individuals who were willing to cooperate with the investigator and research staff, apply the test product according to the protocol, and complete the full course of the survey
Individuals who agree to avoid excessive amounts of UV light as much as possible for the duration of the survey
Male volunteers to be clean shaven

The participant should not imbibe more than 14 units of alcohol per week for the duration of the study.

The participant is a non-smoker who has not used nicotine or nicotine-containing products for at least 1 month prior to screening and for the duration of the study.

### Exclusion Criteria

Prior treatment with retinoids unless the treatment ended at least one month for topical and 3 months for oral preparations prior to enrolment in this study.

Significant acute, unstable, chronic (including symptoms of headaches, dysphoria, fatigue, dizziness, blurred vision, insomnia, rhinorrhoea, nausea, vomiting, abdominal pain, diarrhoea, constipation, menopausal hot flashes/night sweats, or clinically significant premenstrual syndrome) or recurring medical conditions as judged by the investigator.

Clinically significant psychiatric, cardiovascular, respiratory, hepatic, renal, gastrointestinal, endocrine, neurological (particularly myasthenia gravis), immunological, or haematological disease or abnormality, as determined by the study physician.

The participant has a known sensitivity to DIM, or a history of any significant allergy/intolerance that required treatment/intervention.

Currently participating in another study with an investigational or non-investigational drug or device, or has participated in another clinical trial within the 3 months prior to signing the ICF.

Prior treatment with chemotherapy.

Currently taking regular prescribed concurrent medications or hormones (other than oral contraception (which is of a stable dose for at least the last 3 months), or vaccinations, or using over-the-counter medications (except for occasional over the counter analgesics (a maximum of 2 g paracetamol per day is permissible), cold remedies and anti-histamines) from screening and for the duration of the study.

Pregnant or lactating females.

Self-reported history of alcohol, narcotic, benzodiazepine, or other substance abuse or dependence within the 12 months preceding screening.

Underlying diseases or other dermatologic conditions that require the use of interfering topical or systemic therapy such as, but not limited to, atopic dermatitis, perioral dermatitis or rosacea.

Beard or facial hair which might interfere with study assessments.

Use of tanning booths or other light devices within 2 weeks prior to or planned used during the study.

Alpha hydroxy acid products, medicated shaving creams, astringents and preparations with alcohol from screening and for the duration of the study.

Any condition that, in the investigator's opinion, compromises the participant's ability to meet protocol requirements or to complete the study.
History of skin cancer
Individuals who declare to be deprived of their freedom by administrative or legal decision or who are under guardianship
Individuals who cannot be contacted by telephone in case of emergency

### Product

### Product Supply

Product will be supplied to the volunteers by iiaa Ltd., all of which will have been QA monitored and QC tested/released according to EU and UK standards. The market survey manager will make a record of the batch number and expiry date for all cosmetic products allocated to each tester and of any damaged or unusable product.

### Product Storage

All marketing survey product will be stored securely and supplied by the survey manager complete with the tamper evidence seals in place and within the product expiry listed on each pack.

### Randomisation

An independent party will create a randomisation code and label the 4 different products.
Group 1:3 subjects with acne vulgaris will receive topical emugel containing 1% DIM
Group 2:3 subjects with acne vulgaris will receive topical cream containing 1% DIM
Group 3:3 subjects with acne vulgaris will receive topical emugel containing 2.5% DIM
Group 4:3 subjects with acne vulgaris will receive topical cream containing 2.5% DIM
Group 5:3 subjects with atopic dermatitis will receive topical emugel containing 1% DIM
Group 6:3 subjects with atopic dermatitis will receive topical cream containing 1% DIM
Group 7:3 subjects with atopic dermatitis will receive topical emugel containing 2.5% DIM
Group 8:3 subjects with atopic dermatitis will receive topical cream containing 2.5% DIM

### Potential Benefits and Risks

### Potential Benefits

Benefits include an improvement in acne vulgaris and atopic dermatitis severity, as well as reduced redness and reduced trans epidermal water loss may be observed.

### Potential Risks

Topical DIM has been reviewed by a toxicologist and judged safe for topical use up to 2.5%. The assessment is considered to be very low risk. The assessments of skin function are not invasive and therefore of minimal risk.

### Ethics

Ethics committee approval is not required for cosmetic products. The research protocol is conducted in accordance with the Declaration of Helsinki, and with UK and EU standards of good practice, applicable government regulations of cosmetic products, policies and procedures.

### Product Safety

### Reactions During the Survey

All products undergoing market surveys are classed as cosmetic products under both UK and EU law. The pre checks for all participants ensure that no one with a pre-existing systemic condition or acute or chronic facial dermatosis, other than those been assessed, are allowed to take part in the survey. Furthermore, it is essential that participants do not stop taking prescribed medication to be able to take part in the survey. In addition to these pre checks all participants will be provided with information regarding possible reactions, worsening symptoms or psychological distress. They will be provided with a support contact number that they can call at all times during working hours, and will have immediate access to the marketing survey manager supported by a technical pharmacist in the unlikely event that anyone has a reaction to the cosmetic product being assessed. Should reactions occur after hours, they will have been provided with relevant information to take to their local NHS A&E.

All unusual responses to the cosmetic product, illness or experience that develops or worsens during the course of the assessment will be checked by the survey manager and, if necessary, checked remotely by the consultant pharmacist. The participant will then be advised either to continue with the survey or to stop using the cosmetic product as a safety precaution. All reactions will be monitored, recorded and managed according to the in-house iiaa Ltd standard operating procedure for such reactions.

### Post Assessment Product Reaction

Any unresolved reaction to the cosmetic product will be followed up by the survey manager until the events are resolved, or the reaction is otherwise explained.

At the last scheduled visit, the survey manager will instruct the subject to report any subsequent events that the subject, or the subject's personal physician, believes might reasonably be related to the participation in this marketing assessment.

### Recording of Reactions

Upon each contact with the participant, the survey manager will seek information on any reactions by specific questioning provided by the adverse reaction form.

### Example 9: Topical Administration Increases Maximal Plasma Concentration (Cmax) and Dermal Bioavailability (AUC) of DIM in the Skin

The objective of this study was to determine the pharmacokinetic (PK) profile of a diindolylmethane (DIM) formulation in skin following single and multiple doses of DIM, administered cither by topical route or by oral route. The DIM topical formulation for this study was an exemplary formulation prepared according to Example 1 and/or Example 2. The DIM oral formulation for this study is prepared according to U.S. Pat. No. 9,180,117.

Participants were grouped into (a) Group 1: DIM administered by oral route and (b) Group 2: DIM administered by topical route. Participants were were administered, orally or topically, respectively, a single 10 mg dose of the DIM formulation. These participants were not exposed to DIM earlier and thus were naïve subjects. Mean dermal Cmax for the topical administration was assessed and compared to mean dermal Cmax for the oral administration.

The dermal bioavailability of DIM was derived from AUC after the single 10 mg dose, topically or orally, in the naïve subjects. Mean dermal AUC for the topical administration was assessed and compared to mean dermal AUC for the oral administration.

### Example 10: Topical Administration Increases Maximal Plasma Concentration (Cmax) and Dermal Bioavailability (AUC) of DIM in the Skin

A limited pilot clinical study was conducted to assess the effectiveness and tolerability of topical DIM in patients with acne, atopic dermatitis and psoriasis, using either a cream or an emulgel, at either 1% or 2.5% concentrations.

### Method

### I. Participants, Procedure and Product

Twenty-six men and women, aged 18 to 68 years (average 36 yrs), were recruited for this trial. Participants were healthy, from a range of ethnic groups. 5 participants had moderate facial acne vulgaris (20 to 50 inflammatory lesions/papules on the face, excluding the nose), 13 had moderate atopic dermatitis, 3 had psoriasis and 5 had other skin conditions including hyperpigmentation of unknown cause and stasis dermatitis.

Participants were excluded if they had used oral retinoids in the 3 months prior to screening, topical retinoids in the one month prior to screening, or were pregnant or lactating. If on hormonal contraception the prescription needed to remain the same over the 3 months prior to screening and for the duration of the trial. The use of nicotine was prohibited in the month before screening, and the duration of the trial.

For 2 weeks prior to the onset of testing and for the duration of the trial, participants were instructed not to use prescribed medication, topical medication and/or self-tanning products, have any facial procedures/facials/chemical peels/dermabrasion, avoid excessive UV light exposure, take food supplements except for medically prescribed supplements such as iron or vitamin B12, or drink more than 14 units of alcohol per week. They were also required to not make any changes to their skincare routine. The day before testing they were allowed to follow their normal skincare routine, however on the day of testing, they were instructed to only wash their face/neck or affected areas with water and not wear any cosmetics.

All participants were randomly assigned to use either 1% cream, 1% emulgel, 2.5% cream or 2.5% emulgel. Participants were required to apply the product to affected areas twice daily. All subjects provided informed consent before participation.

### II. Assessment

This study was conducted over 16 weeks, with assessments at baseline (week 0) and weeks 4, 8 and 16. Assessment consisted of physiological measurements, imaging, clinical assessments and questionnaires. Where possible similar affected areas in each individual were left untreated for a comparison. Control sites were measured in all participants.

### Physiological Measurements

The Courage & Khazaka™ equipment was used to measure properties of the skin. Courage & Khazaka™ are worldwide leaders in the production of dermatology testing devices.

Hydration in the stratum corneum is assessed with a Corneometer® which measures the capacitance of the di-electric properties of the stratum corneum. Hydration is measured in arbitrary units (AU) between 0 and 120, with very dry being <30, dry being 30-40 and normal >40.

Transepidermal water loss (TEWL) is assessed with a Tewameter® which uses open-chamber evaporimeters to measure TEWL. This together with the electrical method used by the Corneometer are the preferred techniques for measuring the water balance in the stratum corneum. TEWL is measured in g/h/m², with values >20 defined as increased.

Skin elasticity is assessed with a Cutometer®, which uses suction methods to deform skin and a non-contact optical measurement system to measure the deformity and recoil, which are used to calculate the elastic and viscoelastic properties of skin. There are several assessment parameters. We used R2 which measures the proportion between the amplitude after suction (Uf) and the ability to recover to the initial position (Ua). This is expressed as Ua/Uf (relaxation/suction) and is reported as a %. The higher the % the better.

Melanin and erythema (haemoglobin) are assessed with a Mexameter®, which emits 3 specific wavelengths of light and measures the reflectance of skin (light reflected back). Erythema is grouped into: none <170, minimal 170-330, diffuse redness 330-450, high 450-750 and extreme >570. Melanin varies per skin type. Both erythema and melanin are measured with arbitrary units (AU), between 0-999.

Sebum levels are assessed with the Sebumeter®, which uses grease spot photometry. Sebum is measured in μg/cm². On the forehead and t-zone, a value of <100 is defined as dry/less sebum, 100-180 as normal, and >180 as oily.

### Imaging

High-resolution imaging of the skin surface with the Courage and Khazaka™ Visioscan®, a unique high resolution. UVA light video camera. This allows assessment of wrinkle depth, skin smoothness and skin roughness.

Imaging of the full face using the Canfield® Visia equipment. This uses IntelliFlash®, cross-polarized and UV lighting to record and measure surface and subsurface skin conditions.

### Clinical Assessments

### Atopic Dermatitis—Eczema Area and Severity Index (EASI)

The EASI is a validated scale and can be used to reliably assess the severity and extent of atopic dermatitis. Four disease characteristics of atopic dermatitis (erythema, oedema and/or papulation, excoriations, and lichenification) are assessed for severity by the investigator on a scale from 0 to 3, with 0 being absent, 1 mild, 2 moderate and 3 severe. The scores are added up for each of the four body regions (head/neck, trunk, upper extremities and lower extremities), and total scores are multiplied by a multiplier which differs per area. The total EASI score ranges from 0 to 72 points, with the highest score indicating worse severity of atopic dermatitis. The classification of the EASI results: clear=0, almost clear=0.1-1.0, mild=1.1-7.0, moderate 7=0.1-21.0, severe=21.1-50.0 and very severe=50.1-72.

### Acne—Clinical Erythema Index (CEI)

The CEI is a quantitative method for assessing the degree and extent of erythema which is one of the features of moderate and severe acne. The outcome is measured in each of four quadrants of the face. For each of the quadrants, a score is given on an ordinal 5-point scale, with values ranging from 0 (none) to 4 (very severe). The total CEI score is a sum of the five quadrants, giving a total score from 0 to 20.

### Acne—Comprehensive Acne Severity Scale (CASS)

The CASS is used to assess the severity of acne present on the face and/or upper back and chest. It consists of an ordinal 6-point scale, with values ranging from 0 (clear) to 5 (very severe).

### Acne—Global Score for Inflammatory and Non-Inflammatory Lesions

Although various investigators have proposed acne severity grading schemata based upon counts and position on various quadrants of the body, none have been thoroughly validated. In this trial the number of non-inflammatory (NI) and inflammatory (I) lesions was counted, on the face (divided into 5 areas), the chest and the upper back. This approach has been accepted by the FDA in registration studies for Adapalene Cream 0.1 & 0.5% and Dapsone Gel 5%.

### Acne—Cardiff Acne Disability Index (CADI)

This is a short, five-item questionnaire derived from the longer Acne Disability Index that is designed for use in adolescents and young adults to assess acne-related quality of life impairment. It is self-explanatory and can be simply handed to the patient who is asked to complete it without the need for detailed explanation. It is usually completed in one minute. A review by Abdelrazik et al., concluded that the questionnaire is reliable for measuring the impact of acne on the subject, but that no studies to date have yet calculated the smallest CADI score change required for the subject to have experienced a positive benefit from treatment. The CADI score is calculated by summing the score of each question resulting in a possible maximum of 15 and a minimum of 0. The higher the score, the more the quality of life is impaired.

### Questionnaire

A self-assessment questionnaire was administered after initial application of the product, and at the end of weeks 1, 2, 4, 8, 12 and 16.

### III. Statistical Analysis

### Subject Characteristics

Differences in skin parameters, including stratum corneum hydration, elasticity, erythema, melanin, TEWL and sebum production were analysed using one-way ANOVA. This analysis evaluated the effects of treatment group and time (Weeks 0, 4, 8, and 16). Additionally, pooled two-sampled t-tests were conducted to compare differences between groups at each time point.

### Normality Testing

The normality of the data sets was evaluated using the Data Analysis Toolpak in Microsoft Excel to guide the selection of appropriate statistical methods. Levene's test showed that all data sets followed a normal distribution, enabling the use of one-way ANOVA for comparing independent groups.

ANOVA and t-Tests

Each group included both treatment and control areas on the skin, and comparisons between these areas within each group were made using one-way ANOVA. Additionally, t-tests were applied to assess differences across time points (Weeks 0, 4, 8, and 16) within each group.

The effects of Cream versus Emulgel formulations on skin parameters were also compared. One-way ANOVA was used to analyse differences between the Cream and Emulgel formulations within each group at various time points, followed by independent t-tests to assess changes between specific time periods.

Additionally, comparisons between DIM concentrations (1% vs. 2.5%) were conducted to assess whether different concentrations had an effect on skin parameters in each group. One-way ANOVA was used to evaluate differences between the 1% and 2.5% formulations within each group across the time points, with independent t-tests performed to compare differences between each time period.

### IV. Demographics

A total of 26 participants were included in the trial. All participants completed 8 weeks, and 8 completed the full 16 weeks. Table 1 shows the demographics of participants in the trial.

While we intended to recruit only participants with atopic dermatitis or acne, this was an exploratory trial so we also included other skin conditions. These included 3 people with psoriasis, 2 people with stasis dermatitis (other) and 3 with hyperpigmentation of unknown cause (other).

TABLE 1
Demographics of each group and all groups combined

| Group | Sample size | Average Age (Range) | Female | Male |
|---|---|---|---|---|
| Combined group | 26 | 36 years (18-68) | 17 | 9 |
| Atopic dermatitis | 13 | 36 years (18-68) | 8 | 5 |
| Acne | 5 | 24 years (19-28) | 4 | 1 |
| Psoriasis | 3 | 40 years (23-56) | 3 | 0 |
| Other | 5 | 46 years (36-59) | 2 | 3 |

### Results and Discussion

### Atopic Dermatitis

### Physiological Measurements

At baseline the TEWL of affected areas was abnormally high while in control areas it was normal (37.15 vs 16.62 g/h/m², p=0.001), with 10 out of 12 participants (83.3%) having an abnormal TEWL in affected areas. Skin was very dry in affected areas compared to marginally dry in control areas (hydration: 17.41 vs 35.29 AU, p=0.0001) and erythema was higher in affected areas than control areas (390.17 vs 274.96 AU, p=0.0005). Values are shown in table 2. These changes are in keeping with the pathophysiology of atopic dermatitis with a disrupted skin barrier, dehydration and inflammation.

Treatment led to a 27.3% reduction in TEWL from week 0 to 4 (p=0.040), and a 38.7% reduction from week 0 to 16 (approaching significance, p=0.054). By week 16 average TEWL had decreased from very high to close to the normal range after treatment. Hydration increased by 72.9% from week 0 to 8 (p=0.005) and 66.7% from week 0 to 16 (p=0.013) in treated skin. Erythema and melanin appeared to be decreased in the treated group, however these changes were not significant. Elasticity remained unchanged. We intended to compare changes in treated vs untreated skin, however compliance was poor with participants being so excited with the improvement experienced that they used the product on all affected areas, so these results are not included. In the control group, erythema appeared to decrease, although not significant; while TEWL, hydration, melanin and elasticity remained unchanged.

TABLE 2
Measurement of various skin parameters of treated and control
skin at weeks 0 (baseline), 4, 8 and 16 in atopic dermatitis

| | | Week 0 | Week 4 | Week 8 | Week 16 |
|---|---|---|---|---|---|
| Sample | Participants | 13 | 13 | 13 | 4 |
| size (n) | Treated sites | 22 | 22 | 22 | 9 |
| | Control sites | 13 | 13 | 13 | 4 |
| TEWL | Treated | 37.15 | 27.01* | 30.38 | 22.78** |
| (g/h/m²) | Control | 16.62 | 14.46 | 17.72 | 16.36 |
| Hydration | Treated | 17.41 | 24.46 | 30.09*** | 29.02**** |
| (AU) | Control | 35.29 | 36.46 | 37.49 | 40.05 |
| Erythema | Treated | 390.17 | 358.95 | 362.71 | 349.83 |
| (AU) | Control | 274.96 | 260.69 | 261.18 | 216.67 |
| Melanin | Treated | 311.05 | 272.32 | 282.65 | 155.00 |
| (AU) | Control | 128.43 | 107.37 | 115.07 | 117.34 |
| Elasticity | Treated | 75.26 | 78.57 | 80.36 | 75.17 |
| (Ua/Uf, %) | Control | 82.10 | 80.22 | 80.37 | 85.78 |

TEWL = trans-epidermal water loss;
Ua/Uf = relaxation/suction;
*week 0 vs week 4, p = 0.040;
**week 0 vs week 16, p = 0.054;
***week 0 vs week 8, p = 0.005;
****week 0 vs week 16, p = 0.013

### Images

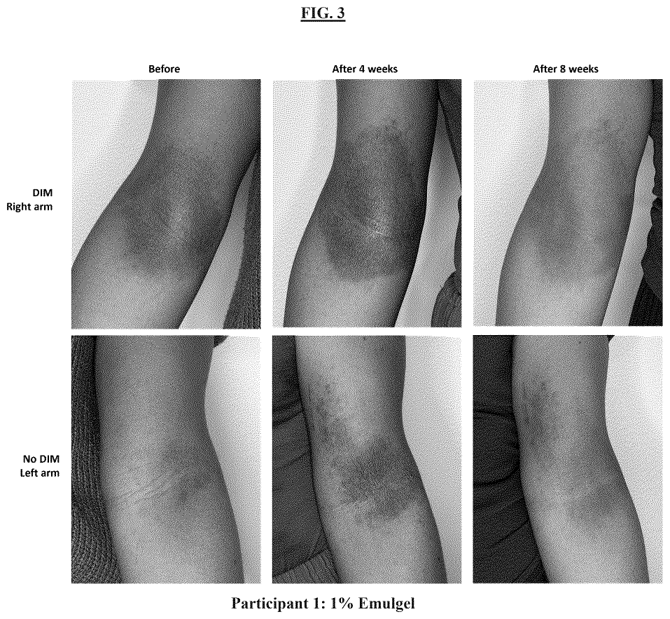

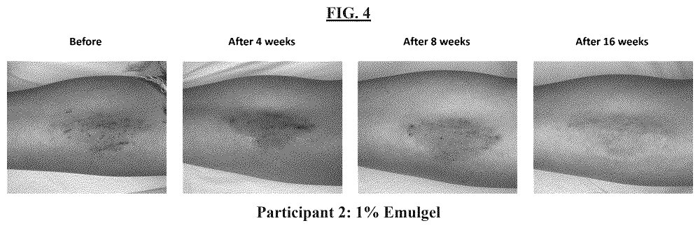

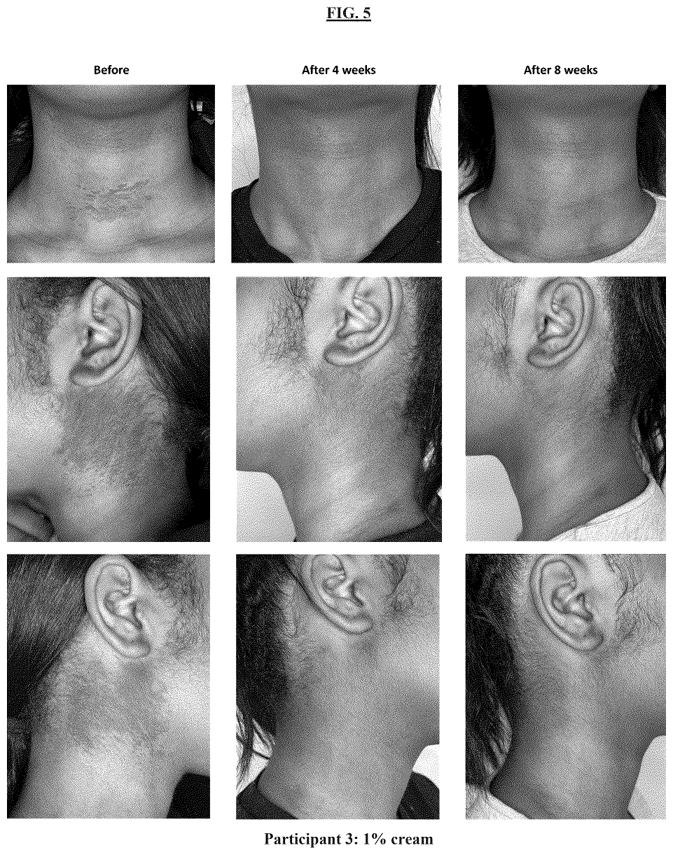

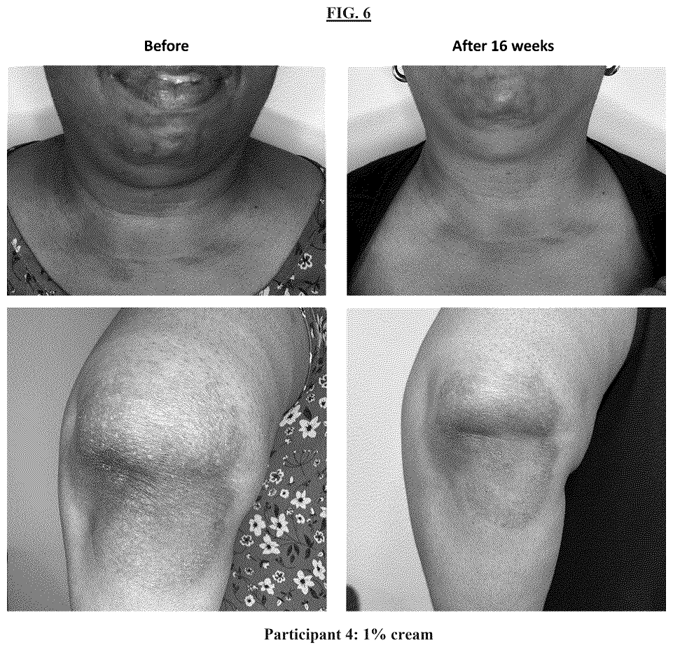

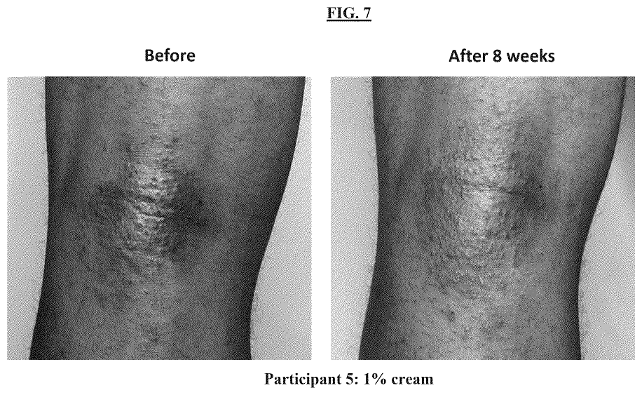

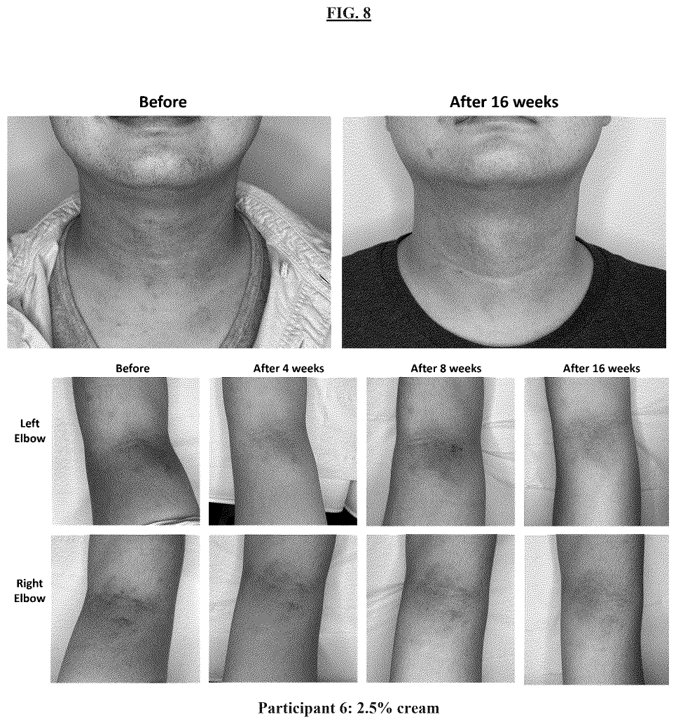

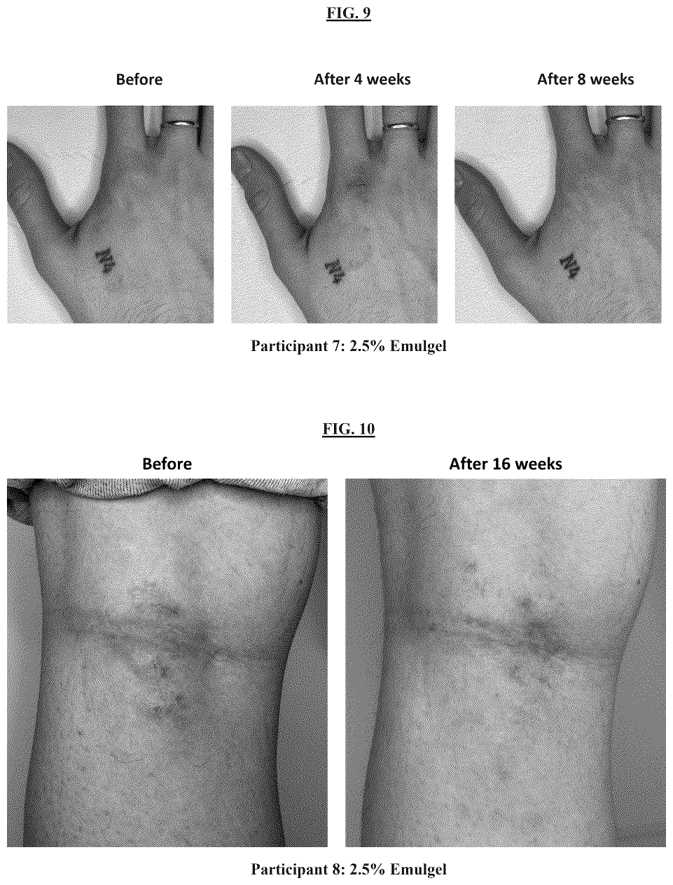

The results of atopic dermatitis patients (Patients 1-8), using Emugel topical formulations containing 1% or 2.5% DIM, or using cream formulations containing 1% or 2.5% DIM., are visually assessed through before and after photographs, were illustrated in FIGS. 3-10. Images show an improvement in the severity of atopic dermatitis with increased smoothness, decreased excoriation, decreased redness and decreased pigmentation over time. They also show healing in the shin area, which is an extremely difficult area to heal as a result of poor blood supply, despite excessive scratching.

### Questionnaire Results

Key results related to skin health:
100% strongly agreed—after 16 weeks of continuous use pigmentation decreased (n=3)
100% agreed-after 16 weeks of continuous use hydration improved (n=3)

### Discussion

At the start of the trial, affected skin showed evidence of a disrupted skin barrier, dehydration and inflammation in keeping with the pathophysiology of atopic dermatitis, while unaffected skin showed normal TEWL with only marginal dryness indicating a healthy skin barrier.

As a result of treatment with topical DIM over the course of the trial, TEWL decreased and hydration increased in affected skin, indicating improvement in the skin barrier. It is likely that if the sample size were larger we would have seen a reduction in erythema levels, indicating decreased inflammation. These improvements are supported by the changes seen in the before and after photos, and the participant testimonials.

### Acne

### Physiological Measurements

At baseline the affected areas had higher TEWL than control areas (20.26 vs 16.20 g/h/m², p=0.029), with 2 out of the 5 participants having an abnormal TEWL in affected areas. Erythema was higher in affected areas (affected vs control: 409.48 vs 223.30 AU, p=0.0001), and elasticity was lower in affected areas (affected vs control: 70.90 vs 89.97%, p=0.007). Melanin appeared higher but this was not significant. Sebum levels in affected skin were in the normal range (there were no control areas for sebum). See values in table 3.

From week 0 to 4 there was a 12.5% reduction in TEWL approaching significance (p=0.057), and a 57.7% reduction in sebum levels (p=0.001). Sebum remained low at week 8 (p=0.001, week 0 vs 8). From week 0 to 16, there was a 33.3% reduction in TEWL (p<0.0001), a 52.6% increase in hydration approaching significance (p=0.074), a 26.8% decrease in erythema (p=0.013) and a 65.0% reduction in sebum (p=0.043), however it is important to bear in mind that there was only 1 participant (with 4 measurement sites) who reached 16 weeks (Table 3). While there appears to be a non-significant decrease in melanin from week 8 to 16, the small sample size needs to be considered. There was no change in elasticity. In control areas, there was a non-significant decrease in hydration (as opposed to the increase seen in treated areas), and no change in any other parameters.

TABLE 3
Measurement of various skin parameters of treated and control
skin at weeks 0 (baseline), 4, 8 and 16 in acne vulgaris

| | | Week 0 | Week 4 | Week 8 | Week 16 |
|---|---|---|---|---|---|
| Sample | Participants | 5 | 5 | 5 | 1 |
| size (n) | Treated sites | 20 | 20 | 20 | 4 |
| | Control sites | 5 | 5 | 5 | 1 |
| TEWL | Treated | 20.26 | 17.73* | 18.68 | 13.52** |
| (g/h/m²) | Control | 16.20 | 14.62 | 15.60 | 10.52 |
| Hydration | Treated | 38.88 | 45.30 | 43.34 | 59.31*** |
| (AU) | Control | 34.73 | 26.28 | 27.07 | 28.73 |
| Erythema | Treated | 409.48 | 378.93 | 396.88 | 299.58**** |
| (AU) | Control | 223.30 | 207.97 | 207.57 | 198.83 |
| Melanin | Treated | 223.01 | 240.98 | 235.02 | 110.89 |
| (AU) | Control | 165.80 | 160.60 | 163.03 | 118.17 |
| Elasticity | Treated | 70.90 | 70.49 | 68.12 | 75.05 |
| (Ua/Uf, %) | Control | 89.97 | 89.96 | 87.58 | 83.95 |
| Sebum | Treated | 128.70 | 54.50^{#} | 54.80^{##} | 45.00^{###} |

| | | |
|---|---|---|
| (μg/cm²) | Control | No measurements were taken |

TEWL = trans-epidermal water loss;
Ua/Uf = relaxation/suction;
*week 0 vs week 4, p = 0.057;
**week 0 vs week 16, p < 0.0001;
***week 0 vs week 16, p = 0.074;
****week 0 to week 16, p = 0.013;
^{#}week 0 vs week 4, p = 0.001;
^{##}week 0 vs week 8, p = 0.001;
^{###}week 0 vs week 16, p = 0.043.

### Clinical Assessments

From week 0 to 8, the CEI improved by 40% (p=0.012), the CASS score improved by 71.4% (p=0.001) and the CADI improved by 55.0% (p=0.025). From week 0 to 8, there was also a reduction in the number of papules and pustules (inflammatory lesions) from 30.4 to 15.0, however this was not significant. Scores are shown in table 4.

TABLE 4
Clinical assessment scores at weeks 0 (baseline),
4, 8 and 16 in participants with acne vulgaris

| | Week 0 | Week 4 | Week 8 | Week 16 |
|---|---|---|---|---|
| Sample size | 5 | 5 | 5 | 1 |
| CEI | 12.00 | 8.00 | 7.20* | 8.00 |
| CASS | 2.80 | 1.30 | 0.80** | 3.00 |
| CADI | 8.00 | 6.00 | 3.60*** | 11.00 |
| Papules and pustules | 30.40 | 20.00 | 15.00 | 26.00 |

CEI = Clinical erythema index;
CASS = Comprehensive Acne Severity Scale;
CADI = Cardiff Acne Disability Index;
*week 0 vs week 8, p = 0.012;
**week 0 vs week 8, p = 0.001;
***week 0 vs week 8, p = 0.025

### Images

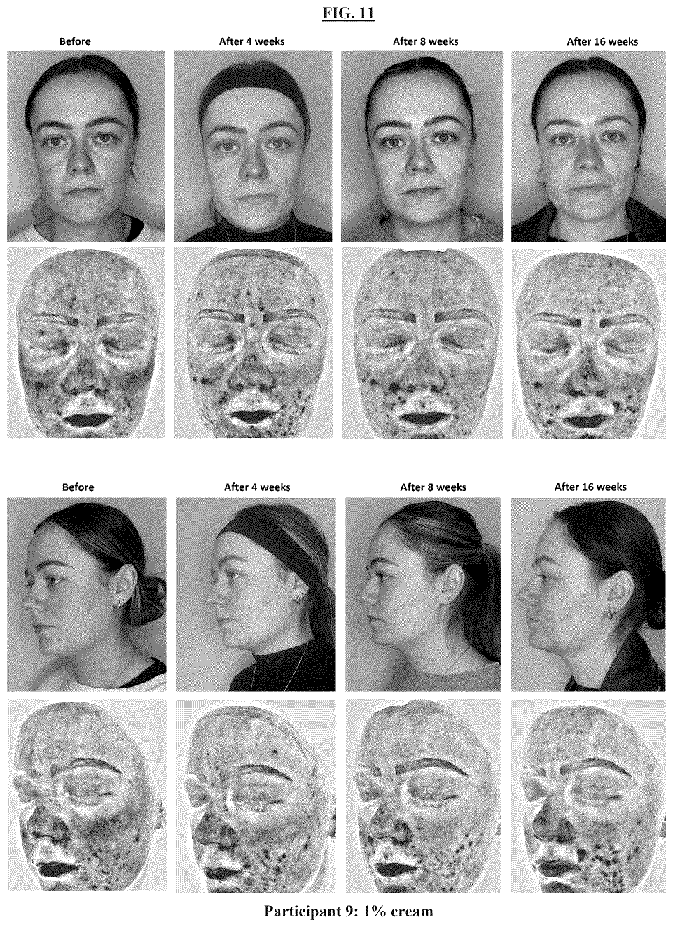

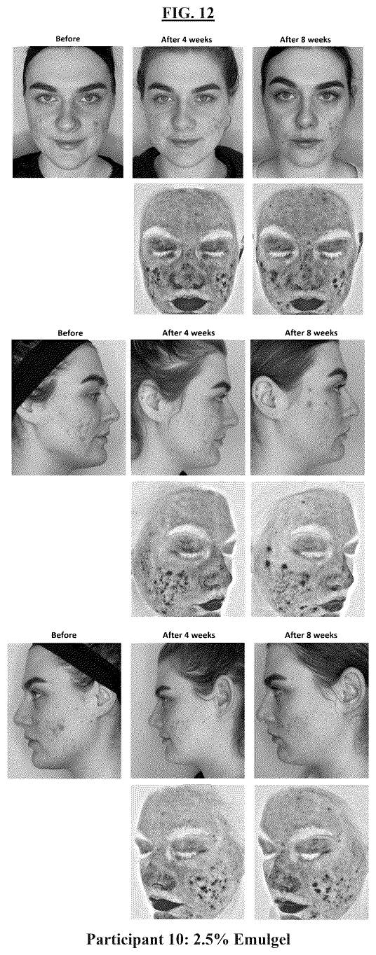

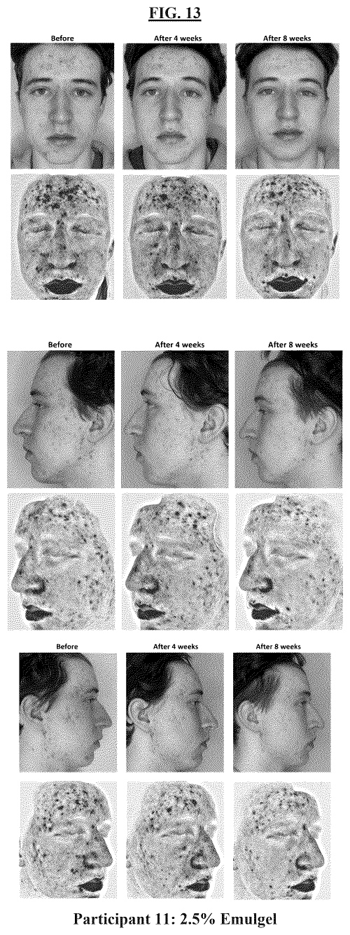

The results of atopic dermatitis patients (Patients 1-8), using Emugel topical formulations containing 1% or 2.5% DIM, or using cream formulations containing 1% or 2.5% DIM., are visually assessed through before and after photographs, were illustrated in FIGS. 11-13.

### Questionnaire Results

Key results related to skin health:
100% agreed—the product instantly hydrates (n=4)
100% agreed—the product calms the skin (n=4)
100% agreed—the product improves pigmentation after 1 week (n=3)
100% agreed—the product improves skin redness after 1 week (n=4)
100% agreed—the product helps keep my skin looking healthier (n=4)
100% agreed—the product improves skin hydration after 2 weeks (n=4)
100% agreed—the product helps keep my skin looking healthier (n=4)
100% agreed—the product improves skin hydration after 4 weeks (n=4)
100% agreed—the product Improves skin texture after 4 weeks (n=4)
100% strongly agreed—after 8 weeks continuous use redness decreased (n=4)
100% strongly agreed—after 8 weeks continuous use skin blemishes decreased (n=4)
100% strongly agreed—after 8 weeks continuous use skin texture improved (n=4)
100% strongly agreed—after 8 weeks continuous use skin looks healthier (n=4)
100% agreed—after 8 weeks continuous use, this product decreased my acne (n=4)
100% agreed—after 8 weeks continuous use, this product improved hydration (n=4)
100% agreed—after 8 weeks continuous use, this product improved acne scarring (n=4)

### Discussion

At the start of the trial, affected skin showed increased erythema indicating inflammation, marginally higher TEWL indicating a slightly disrupted skin barrier, and lower elasticity which has been associated with increased facial pores, compared to control skin. While not significant, melanin appeared higher suggesting post-inflammatory pigmentation. 5 participants completed 8 weeks of treatment with topical DIM. TEWL decreased in affected skin, indicating improvement in the skin barrier, and sebum decreased. Clinical measures showed significant improvements in erythema, acne severity and quality of life, and a non-significant decrease in number of inflammatory lesions. Only 1 participant continued to week 16, and showed a further decrease in TEWL and sebum, as well as decreased erythema, indicating continued reduction in inflammation and recovery of the skin barrier. These improvements are supported by the changes seen in the before and after photos with the majority of participants showing decreased general redness, and the participant questionnaires and testimonials showing reported improvement in redness, pigmentation and hydration, and decreased acne severity and scarring over the duration of the trial.

### Psoriasis

### Physiological Measurements at Baseline for all Participants

At baseline the affected areas had lower hydration than control areas (5.20 vs 41.08 AU, p=0.00001). Average TEWL in the affected areas was 30.48 g/h/m² which is in the abnormally high range (>20), and higher than control (30.48 vs 13.22 g/h/m² respectively) however this was not significant. Erythema levels were higher than control (328.26 vs 170.89 AU, p=0.005), and melanin was higher than control (123.19 vs 75.17 AU, p=0.013). This shows that in affected areas TEWL was abnormally high indicating a disrupted skin barrier. Hydration was low, which is commonly associated with a disrupted barrier. Increased erythema indicates inflammation, and increased melanin possible post-inflammatory pigmentation. As the sample size was very small, the results from during the trial are presented as a case series.

### Participant 12

Product used: 1% cream

### Physiological Measurements

### Baseline

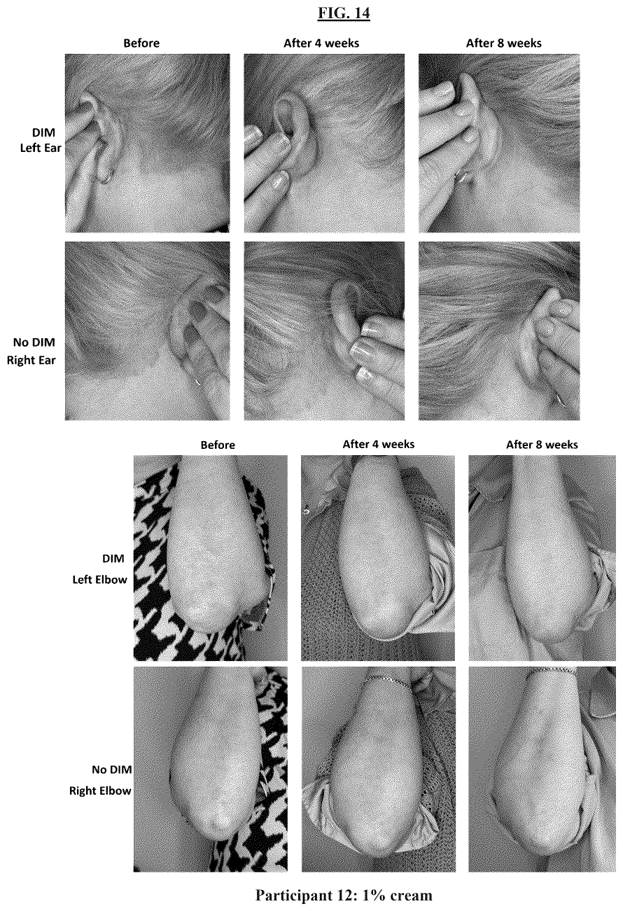

All skin measurements are shown in table 5. TEWL in affected areas behind both ears was high at >30 g/h/m², and both showed very low levels of hydration, erythema was higher than the control and classified as diffuse redness, and melanin levels were higher than the control. All parameters were similar behind left and right ears. Both forearms had a normal TEWL, however hydration was very low, and erythema and melanin higher than the control. All parameters were similar on both forearms. Images, shown in FIG. 14, show that the forearms were much less affected than behind the cars. In the control area, TEWL was normal and hydration marginally low but approaching normal.

### Over the 8 Weeks of the Trial

Behind both ears, TEWL improved with a greater improvement behind the treated left ear (32.00 to 9.31 g/h/m²) than the untreated right ear (37.20 to 15.88 g/h/m²). TEWL in both areas reached normal levels by week 8. Hydration also improved behind both areas, with the treated left having a much greater improvement than untreated right (left improved from 6.50 to 50.63 AU, while right improved from 4.60 to 17.82 AU). Despite the improvement on the right, skin was still very dry at week 8, while the skin on the treated left side showed normal hydration levels. Erythema decreased behind both left and right cars, and melanin decreased marginally behind the treated left ear and increased behind the untreated right car, suggesting that treatment decreased post-inflammatory pigmentation. TEWL improved on the treated left forearm (15.69 to 7.86 g/h/m²) while remaining relatively stable on the untreated right forearm (14.45 to 14.13 g/h/m²). In both areas TEWL was in the normal range throughout the trial. Hydration improved in both areas, with the treated left having a much greater improvement than untreated right (left improved from 8.76 to 31.83 AU, while right improved from 5.65 to 14.28 AU). Despite the improvement on the right, skin was still very dry at week 8, while the skin on the treated left side was only slightly dry.

TABLE 5
Measurement of various skin parameters of treated,
untreated and control skin at weeks 0 (baseline),
4, 8 and 16 for participant 12 with psoriasis

| | | Week 0 | Week 4 | Week 8 |
|---|---|---|---|---|
| TEWL (g/h/m²) | Treated L ear | 32.00 | 9.41 | 9.31 |
| | Untreated R ear | 37.20 | 15.23 | 15.88 |
| | Treated L forearm | 15.69 | 14.04 | 7.86 |
| | Untreated R forearm | 14.45 | 14.60 | 14.13 |
| | Control R wrist | 12.34 | 11.80 | 9.76 |
| Hydration (AU) | Treated L ear | 6.50 | 44.15 | 50.63 |
| | Untreated R ear | 4.60 | 10.12 | 17.82 |
| | Treated L forearm | 8.78 | 37.02 | 31.83 |
| | Untreated R forearm | 5.65 | 12.70 | 14.28 |
| | Control R wrist | 36.10 | 35.87 | 35.67 |
| Erythema (AU) | Treated L ear | 409.50 | 314.33 | 298.83 |
| | Untreated R ear | 432.00 | 329.83 | 286.83 |
| | Treated L forearm | 267.17 | 248.67 | 246.83 |
| | Untreated R forearm | 295.00 | 227.83 | 239.33 |
| | Control R wrist | 177.17 | 159.83 | 144.00 |
| Melanin (AU) | Treated L ear | 102.33 | 121.00 | 98.50 |
| | Untreated R ear | 105.17 | 118.17 | 127.00 |
| | Treated L forearm | 111.83 | 111.33 | 110.83 |
| | Untreated R forearm | 115.00 | 114.50 | 117.17 |
| | Control Wrist | 60.00 | 82.33 | 70.83 |
| Elasticity (Ua/Uf, %) | Treated L ear | 96.00 | 65.30 | 68.05 |
| | Untreated R ear | 95.35 | 100.45 | 100.80 |
| | Treated L forearm | 93.80 | 87.85 | 82.10 |
| | Untreated R forearm | 108.00 | 101.95 | 78.25 |
| | Control Wrist | 72.80 | 64.75 | 71.60 |

TEWL = trans-epidermal water loss;
Ua/Uf = relaxation/suction;
L = left;
R = right.

### Participant 13

Product used: 2.5% Emulgel

### Physiological Measurements

All skin measurements are shown in table 6. At baseline, TEWL in the affected area was extremely high, compared to the control area which was in the normal range. Skin was very dry in affected area and slightly dry in control. From week 0 to 8, TEWL decreased in the affected area from 61.91 to 28.23 g/h/m². Even though TEWL remained high, it improved by 54.4%, suggesting improvement of the skin barrier. In the control skin TEWL decreased by 17.1%, however was in the normal range at baseline. Hydration increased from 7.92 to 10.78 AU in the affected area, while it decreased from 31.38 to 26.63 AU in the control area-showing very dry skin in both affected and control areas. Erythema decreased in both areas: from 306.50 to 213.67 AU in the affected area, and from 189.17 to 142.33 AU in the control area. Melanin and elasticity remained similar.

TABLE 6
Measurement of various skin parameters of treated and control skin
at weeks 0 (baseline), 4 ar 8 for participant 13 with psoriasis

| | | Week 0 | Week 4 | Week 8 |
|---|---|---|---|---|
| TEWL (g/h/m²) | Treated - neck | 61.91 | 32.39 | 28.23 |
| | Control - wrist | 13.12 | 11.22 | 10.88 |
| Hydration (AU) | Treated - neck | 7.92 | 17.82 | 10.78 |
| | Control - wrist | 31.38 | 45.53 | 26.63 |
| Erythema (AU) | Treated - neck | 306.50 | 275.83 | 213.67 |
| | Control - wrist | 189.17 | 178.00 | 142.33 |
| Melanin (AU) | Treated - neck | 113.33 | 121.67 | 140.00 |
| | Control - wrist | 78.83 | 83.17 | 72.67 |
| Elasticity | Treated - neck | 87.50 | 81.40 | 83.60 |
| (Ua/Uf, %) | Control - wrist | 77.70 | 78.75 | 81.95 |

TEWL = trans-epidermal water loss;
Ua/Uf = relaxation/suction.

### Participant 14

Product used: 2.5% cream

### Physiological Measurements

All skin measurements are shown in table 7. At baseline, in both affected elbows TEWL was abnormally high and hydration extremely low, while in the control both were in the normal range. Both melanin and erythema were higher than in the control area, while elasticity was similar. From 0 to 16 weeks, on the right elbow, TEWL increased from 20.50 to 40.41 g/h/m² as did hydration from 1.17 to 21.73 AU. On the left elbow, TEWL decreased from 31.59 to 26.23 g/h/m² and hydration increased from 1.82 to 7.53 AU. Melanin decreased on both elbows and erythema increased. It is possible that the thick psoriasis plaques at baseline prevented accurate measurement of erythema. Interestingly, TEWL also increased in the right wrist which was the control, starting with normal TEWL of 14.21 g/h/m² and increasing to an extremely high TEWL of 79.08 g/h/m², a far greater increase than seen in the right elbow. This suggests that TEWL increased in the right arm in general for an unknown reason, with the product decreasing the deterioration. All other variables in the control area remained similar between 0 and 16 weeks.

TABLE 7
Measurement of various skin parameters of treated and control skin at
weeks 0 (baseline), 4, 8 and 16 for participant 14 with psoriasis

| | | Week 0 | Week 4 | Week 8 | Week 16 |
|---|---|---|---|---|---|
| TEWL | Treated R elbow | 20.50 | 45.07 | 63.75 | 41.41 |
| (g/h/m²) | Treated L elbow | 31.59 | 52.60 | 45.82 | 26.23 |
| | Control R wrist | 14.21 | 26.07 | 45.85 | 79.08 |
| Hydration | Treated R elbow | 1.17 | 4.47 | 10.48 | 21.73 |
| (AU) | Treated L elbow | 1.82 | 7.05 | 11.62 | 7.53 |
| | Control R wrist | 46.07 | 51.98 | 59.95 | 49.65 |
| Erythema | Treated R elbow | 264.33 | 560.67 | 546.33 | 373.83 |
| (AU) | Treated L elbow | 323.33 | 499.83 | 509.33 | 384.50 |
| | Control R wrist | 146.33 | 149.83 | 158.00 | 151.83 |
| Melanin | Treated R elbow | 164.67 | 86.83 | 126.17 | 117.17 |
| (AU) | Treated L elbow | 150.00 | 96.00 | 99.83 | 117.50 |
| | Control R wrist | 86.67 | 87.83 | 88.67 | 72.33 |
| Elasticity | Treated R elbow | 73.75 | 98.10 | 88.75 | 88.6 |
| (Ua/Uf, %) | Treated L elbow | 110.30 | 75.55 | 57.75 | |
| | Control R wrist | 85.15 | 87.35 | 87.70 | 86.9 |

TEWL = trans-epidermal water loss;
Ua/Uf = relaxation/suction;
L = left;
R = right.

### Questionnaire Results for all 3 Participants

Key results related to skin health:
100% strongly agree—after 1 week continuous use, psoriasis improved (n=3)
100% strongly agree—after 1 week continuous use, hydration improved (n=3)
100% strongly agree—after 1 week continuous use, skin looked healthier (n=3)
100% strongly agree—after 2 weeks continuous use, psoriasis improved (n=3)
100% strongly agree—after 2 weeks continuous use, hydration improved (n=3)
100% strongly agree—after 2 weeks continuous use, skin looked healthier (n=3)
100% strongly agree—after 4 weeks continuous use, hydration improved (n=3)
100% strongly agree—after 8 weeks continuous use, redness decreased (n=3)
100% strongly agree—after 8 weeks continuous use, psoriasis improved (n=3)
100% strongly agree—after 12 weeks continuous use, psoriasis improved (n=2)
100% strongly agree—after 12 weeks continuous use, redness decreased (n=2)
100% strongly agree—after 12 weeks continuous use, hydration improved, (n=2)
100% strongly agree—after 12 weeks continuous use, skin texture improved (n=2)
100% strongly agree—after 12 weeks continuous use, skin looked healthier (n=2)
100% strongly agree—after 12 weeks continuous use, pigmentation decreased (n=2)

### Discussion

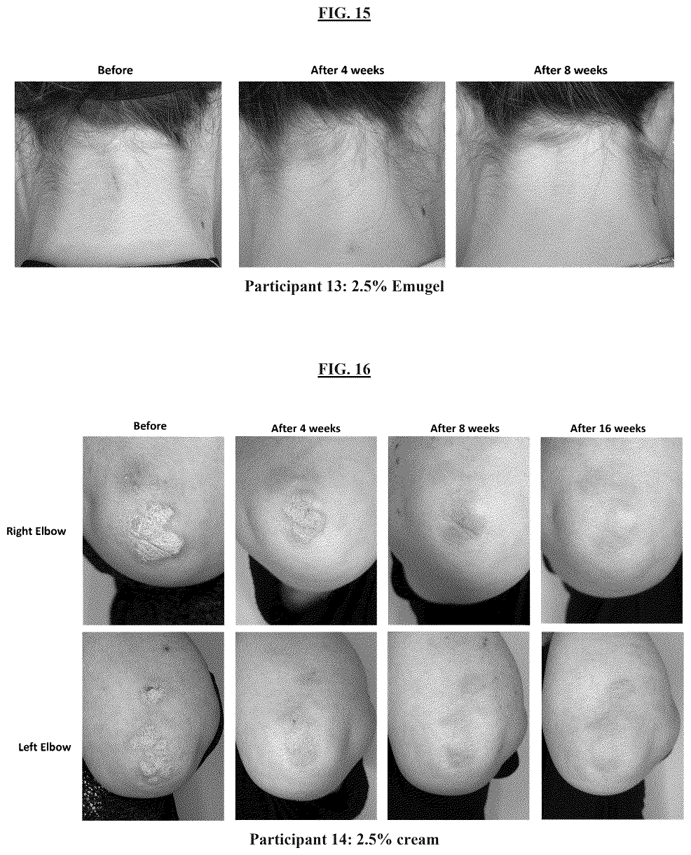

At baseline, in affected areas TEWL was abnormally high indicating a disrupted skin barrier. Hydration was low, which is commonly associated with a disrupted barrier. Increased erythema indicates inflammation, and increased melanin possible post-inflammatory pigmentation. Despite the small sample size, the participants responded to treatment in similar ways. In all participants treatment led to improvements in TEWL and improved hydration, suggesting improved skin barrier function. Treatment led to decreased erythema except in patient 3, which was possibly caused by thick psoriasis plaques at baseline prevented accurate measurement of erythema. Images of the treated areas in all 3 participants showed marked improvement (FIG. 14-16). These visual improvements are in line with improvements in skin measurements.

### Combination of Skin Conditions

### Physiological Measurements

### Baseline

At baseline the affected areas had higher TEWL than control areas (28.07 vs 16.29 g/h/m², p=0.001). Hydration was lower in affected vs control (25.08 vs 34.77 AU, p=0.003), and erythema was higher in affected vs control areas (383.88 vs 255.30 AU, p<0.0001). All values are shown in table 8.

### During the Trial

TEWL decreased by 19.9% from week 0 to week 4 (p=0.039), and from week 0 to week 16 (27.3% reduction, p=0.048). All participants starting with an abnormal TEWL showed a 29.7% reduction by week 4 (p=0.002), and a 40.6% reduction by week 16 (p=0.007). Hydration increased by 33.9% by week 8 (p=0.020) and remained stable to week 16; while erythema decreased by 16.8% by week 16 (p=0.003). Sebum decreased in the treated group, while it increased in the control group—both of these were non-significant. There was also a non-significant increase in elasticity in the treated group, while the control group remained the same. There was no change in melanin in either group, and no change in TEWL, hydration, erythema and elasticity in the control group.

TABLE 8
Measurement of various skin parameters of treated,
untreated and control skin at weeks 0 (baseline),
4, 8 and 16 for participants with all skin conditions

| | | Week 0 | Week 4 | Week 8 | Week 16 |
|---|---|---|---|---|---|
| TEWL (g/h/m²) | Treated | 28.07 | 22.47* | 24.97 | 20.41** |
| | Control | 16.29 | 16.40 | 18.22 | 20.33 |
| Hydration (AU) | Treated | 25.08 | 31.93 | 33.57*** | 33.35 |
| | Control | 34.77 | 33.79 | 34.26 | 37.65 |
| Erythema (AU) | Treated | 383.88 | 356.92 | 366.53 | 319.34**** |
| | Control | 255.30 | 245.05 | 240.08 | 219.61 |
| Melanin (AU) | Treated | 326.06 | 340.10 | 336.53 | 278.19 |
| | Control | 243.01 | 220.53 | 223.52 | 163.17 |
| Elasticity | Treated | 76.47 | 76.54 | 75.71 | 88.17 |
| (Ua/Uf, %) | Control | 82.75 | 82.35 | 81.10 | 81.48 |
| Sebum (μg/cm²) | Treated | 40.91 | 20.82 | 21.94 | 17.43 |
| | Control | 16.62 | 21.00 | 6.54 | 39.20 |

TEWL = trans-epidermal water loss;
Ua/Uf = relaxation/suction;
*week 0 vs week 4, p = 0.039;
**week 0 vs week 16, p = 0.048;
***week 0 vs week 8, p = 0.020;
****week 0 vs week 16, p = 0.003.

### Images

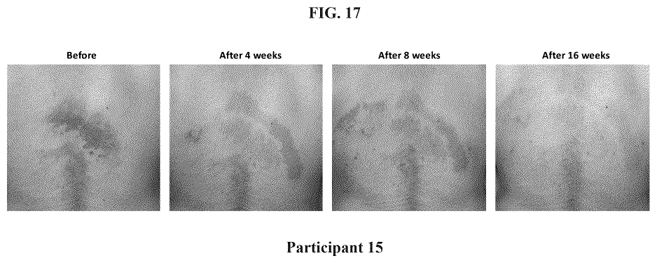

Images showing improvements in both erythema and pigmentation (FIG. 17).

### Questionnaire Results

Key results related to skin health:
88% agree—the product hydrates skin (n=24)
88% agree—the product instantly soothes skin (n=24)
88% agree—the product instantly calms skin (n=24)
88% agree—after 2 weeks continuous use, the product hydrates skin (n=24)
83% agree—after 4 weeks continuous use, the product hydrates skin (n=24)
90% agree—after 16 weeks continuous use, the product hydrates skin (n=11)

### Discussion

All participants, regardless of the skin condition, had higher TEWL, lower hydration and higher erythema in affected skin vs control skin at the start of the trial. This suggests an impaired skin barrier and inflammation. Topical use of DIM led to an improvement in skin barrier function as evidenced by a decrease in TEWL, increased hydration and decreased erythema suggesting improved inflammation over the course of the 16-week trial. Improvements in TEWL were evident from 4 weeks, and improvements in hydration from 8 weeks. Self-assessment questionnaires support the improvements in skin hydration.

### Emulgel vs Cream

### Atopic Dermatitis

6 participants used the cream, while 7 used the emulgel. At baseline, there was no difference between groups.

The use of the emulgel led to a significant increase in hydration in affected areas over the trial (week 8, p=0.029; week 16, p=0.061), while use of the cream lead to a non-significant increase. TEWL also appears to decrease in both groups, however this is not significant. Skin measurements are shown in table 9.

TABLE 9
Measurement of various skin parameters treated by cream and Emulgel
at weeks 0 (baseline), 4, 8 and 16 in atopic dermatitis

| | | Week 0 | Week 4 | Week 8 | Week 16 |
|---|---|---|---|---|---|
| TEWL (g/h/m²) | Cream | 30.78 | 22.86 | 25.86 | 20.13 |
| | Emulgel | 46.34 | 33.00 | 36.90 | 28.09 |
| Hydration (AU) | Cream | 19.50 | 26.95 | 29.41 | 29.36 |
| | Emulgel | 14.39 | 20.88 | 31.07* | 28.32** |
| Erythema (AU) | Cream | 412.64 | 385.64 | 387.00 | 364.97 |
| | Emulgel | 357.70 | 320.41 | 327.63 | 319.56 |
| Melanin (AU) | Cream | 483.41 | 492.29 | 494.99 | 457.56 |
| | Emulgel | 399.30 | 448.43 | 431.57 | 152.67 |
| Elasticity (Ua/Uf, %) | Cream | 73.70 | 81.43 | 80.62 | 73.99 |
| | Emulgel | 77.51 | 74.43 | 79.99 | 77.53 |
| Sebum (μg/cm²) | Cream | 5.29 | 3.43 | 9.57 | 6.50 |
| | Emulgel | 2.43 | 5.57 | 9.71 | 30.00 |

TEWL = trans-epidermal water loss;
Ua/Uf = relaxation/suction;
*week 0 vs week 8, p = 0.029;
**week 0 vs week 16, p = 0.061.

### Acne

2 participants used the cream, while 3 used the emulgel. At baseline, the groups using cream and emulgel were slightly different: the group using the cream had normal hydration levels while the group using the emulgel had very dry skin (cream vs emulgel, 56.83 vs 26.9 AU, p=0.0001); and the group using the cream had TEWL in the normal range while the group using the emulgel had high TEWL (cream vs Emulgel, 18.55 vs 21.39 g/h/m², p=0.057). This suggests that the cream group had a normal skin barrier while the emulgel group had a disrupted skin barrier.

Emulgel led to increased hydration over the trial (week 4, p=0.026; week 8, p=0.058), improving from very dry to marginally dry, while the cream remained unchanged in the normal range. However, one needs to take the different values at the start into consideration. Cream led to decreased TEWL over the trial (week 4, p=0.003; week 8, p=0.028; week 16, p=0.003), while there was no change in response to emulgel. Both cream and emulgel led to decreased sebum over the trial (Cream: week 4, p=0.022, week 8, p=0.076; Emulgel: week 4, p=0.023; week 8, p=0.010). There was no difference between groups at start. All measurements are shown in table 10.

TABLE 10
Measurement of various skin parameters treated by Cream
and Emulgel at weeks 0 (baseline), 4, 8 and 16 in acne

| | | Week 0 | Week 4 | Week 8 | Week 16 |
|---|---|---|---|---|---|
| TEWL | Cream | 18.55 | 13.74* | 14.97** | 13.52*** |
| (g/h/m²) | Emulgel | 21.39 | 20.39 | 21.15 | |
| Hydration | Cream | 56.83 | 54.93 | 53.29 | 59.31 |
| (AU) | Emulgel | 26.90 | 38.88**** | 36.71^{#} | |
| Erythema | Cream | 447.38 | 379.48 | 414.67 | 299.58 |
| (AU) | Emulgel | 384.21 | 378.56 | 385.03 | |
| Melanin | Cream | 397.08 | 442.60 | 437.56 | 131.46 |
| (AU) | Emulgel | 106.96 | 106.56 | 100.00 | |
| Elasticity | Cream | 65.16 | 67.27 | 68.99 | 75.05 |
| (Ua/Uf, %) | Emulgel | 74.72 | 72.64 | 67.54 | — |
| Sebum | Cream | 122.50 | 37.25^{##} | 52.25^{###} | 45.00 |
| (μg/cm²) | Emulgel | 132.83 | 66.00^{####} | 56.50^{\hat{ }} | — |

TEWL = trans-epidermal water loss;
Ua/Uf = relaxation/suction;
*week 0 vs week 4, p = 0.003;
**week 0 vs week 8, p = 0.028;
***week 0 vs week 16, p = 0.003;
****week 0 vs week 4, p = 0.026;
^{#}week 0 vs week 8, p = 0.058;
^{##}week 0 vs week 4, p = 0.022;
^{###}week 0 vs week 8, p = 0.076;
^{####}week 0 vs week 4, p = 0.023;
\hat{ }week 0 vs week 8, p = 0.010.

### Combined

11 participants used the cream, while 15 used the emulgel. At baseline, erythema was higher in cream group compared to emulgel (p=0.011). Both cream and emulgel led to decreased erythema, however this was only significant in the cream group (week 16, p-0.006). Both cream and emulgel led to increased hydration, however this was only significant in the emulgel group (week 4, p=0.052; week 8, p-0.017).

Skin measurements shown in table 11.

TABLE 11
Measurement of various skin parameters treated by cream and Emulgel
at weeks 0 (baseline), 4,8 and 16 in combined skin conditions

| | | Week 0 | Week 4 | Week 8 | Week 16 |
|---|---|---|---|---|---|
| TEWL (g/h/m²) | Cream | 25.48 | 20.69 | 22.82 | 19.25 |
| | Emulgel | 30.57 | 24.19 | 27.04 | 22.72 |
| Hydration (AU) | Cream | 29.54 | 35.59 | 36.43 | 35.35 |
| | Emulgel | 20.78 | 28.40* | 30.81** | 24.60 |
| Erythema (AU) | Cream | 410.87 | 379.18 | 388.90 | 329.60*** |
| | Emulgel | 357.89 | 335.48 | 344.99 | 300.29 |
| Melanin (AU) | Cream | 386.71 | 402.69 | 403.16 | 276.49 |
| | Emulgel | 267.65 | 279.83 | 272.38 | 281.36 |
| Elasticity | Cream | 73.64 | 77.85 | 77.00 | 75.70 |
| (Ua/Uf, %) | Emulgel | 78.09 | 75.74 | 75.39 | 77.46 |
| Sebum (μg/cm²) | Cream | 37.13 | 14.73 | 20.40 | 17.57 |
| | Emulgel | 46.22 | 26.17 | 24.39 | 19.83 |

TEWL = trans-epidermal water loss;
Ua/Uf = relaxation/suction;
*week 0 vs week 4, p = 0.052;
**week 0 vs week 8, p = 0.017;
***week 0 vs week 16, p = 0.006.

Emulgel led to increases in hydration in participants with atopic dermatitis, acne and combined skin conditions, while cream led to non-significant increases in hydration in atopic dermatitis and combined skin conditions. One should note that the group of participants with acne using the cream had higher hydration levels at the start. Cream led to decreased TEWL in acne, even though TEWL was lower to start, as well as non-significant decreases in TEWL in atopic dermatitis. Emulgel also led to non-significant decrease in TEWL in atopic dermatitis. Both cream and emulgel led to a decrease in erythema in participants with all skin conditions, although erythema was higher to start in that group. Both emulgel and cream led to a decrease in sebum in participants with acne. In summary, both cream and emulgel led to improvements in TEWL, hydration, erythema and sebum. However, due to the small sample sizes, it is not possible to make a conclusive comparison.

### 1% Vs 2.5% DIM Concentrations

There was no difference between groups at baseline.

### Atopic Dermatitis

4 participants used the 1% product, while 8 used the 2.5% product.

The 1% product led to a non-significant increase in hydration, while the 2.5% product led to a significant increase in hydration (week 8, p=0.012; week 16, p=0.005). Skin measurements are shown in table 12.

TABLE 12
Measurement of various skin parameters treated
by 1% and 2.5% DIM concentrations at weeks 0
(baseline), 4, 8 and 16 in atopic dermatitis

| | | Week 0 | Week 4 | Week 8 | Week 16 |
|---|---|---|---|---|---|
| TEWL (g/h/m²) | 1% | 24.34 | 19.60 | 22.01 | 15.97 |
| | 2.5% | 45.44 | 31.73 | 35.65* | 28.23** |
| Hydration (AU) | 1% | 21.48 | 35.20 | 34.79 | 30.51 |
| | 2.5% | 13.53 | 19.10 | 28.29 | 27.82 |
| Erythema (AU) | 1% | 416.87 | 407.62 | 414.08 | 388.38 |
| | 2.5% | 375.90 | 329.40 | 328.51 | 319.00 |
| Melanin (AU) | 1% | 719.07 | 676.63 | 677.02 | 503.83 |
| | 2.5% | 283.50 | 335.42 | 320.27 | 237.60 |
| Elasticity (Ua/Uf, %) | 1% | 72.34 | 80.41 | 82.15 | 76.56 |
| | 2.5% | 77.92 | 77.87 | 79.18 | 74.06 |
| Sebum (μg/cm²) | 1% | 8.00 | 5.00 | 13.00 | 6.00 |
| | 2.5% | 1.56 | 4.22 | 7.78 | 30.33 |

TEWL = trans-epidermal water loss;
Ua/Uf = relaxation/suction;
*week 0 vs week 8, p = 0.012;
**week 0 vs week 16, p = 0.005.

### Acne

1 participant used the 1% product, while 4 used the 2.5% product. The 1% product led to a decrease in TEWL (week 4, p=0.054; week 8, p=0.035; week 16, p=0.038), however it is important to note that there was only 1 subject. The 2.5% product also led to a decrease in TEWL, however this was not significant. The 2.5% led to a decrease in sebum (week 4, p=0.003; week 8, p=0.001), while the 1% led to a non-significant decrease. Skin measurements are shown in table 13.

TABLE 13
Measurement of various skin parameters treated by 1% and 2.5%
DIM concentrations at weeks 0 (baseline), 4, 8 and 16 in acne

| | | Week 0 | Week 4 | Week 8 | Week 16 |
|---|---|---|---|---|---|
| TEWL (g/h/m²) | 1% | 19.30 | 14.09* | 13.18** | 14.01*** |
| | 2.5% | 20.50 | 18.64 | 20.06 | — |
| Hydration (AU) | 1% | 48.47 | 52.64 | 50.53 | 59.31 |
| | 2.5% | 36.48 | 43.46 | 41.54 | — |
| Erythema (AU) | 1% | 382.04 | 302.63 | 318.21 | 299.58 |
| | 2.5% | 416.33 | 398.00 | 416.55 | — |
| Melanin (AU) | 1% | 92.04 | 110.96 | 107.33 | 131.46 |
| | 2.5% | 255.75 | 273.48 | 266.95 | — |
| Elasticity (Ua/Uf, %) | 1% | 60.45 | 64.90 | 73.33 | 75.05 |
| | 2.5% | 73.51 | 71.89 | 66.82 | — |
| Sebum (μg/cm²) | 1% | 131.00 | 36.50 | 80.00 | 45.00 |
| | 2.5% | 128.13 | 59.00^{#} | 48.50^{##} | — |

TEWL = trans-epidermal water loss;
Ua/Uf = relaxation/suction;
*week 0 vs week 4, p = 0.054;
**week 0 vs week 8, p = 0.035;
***week 0 vs week 16, p = 0.038;
^{#}week 0 vs week 4, p = 0.003;
^{##}week 0 vs week 8, p = 0.001)

### Combined Skin Conditions

9 participants used the 1% product, while 17 used the 2.5% product. The 1% product led to a decrease in TEWL (week 16, p=0.048), and an increase in hydration (week 16, p=0.024), while the 2.5% product led to a non-significant increase in hydration. Skin measurements are shown in table 14.

TABLE 14
Measurement of various skin parameters treated by
1% and 2.5% DIM concentrations at weeks 0 (baseline),
4, 8 and 16 in combined skin conditions

| | | Week 0 | Week 4 | Week 8 | Week 16 |
|---|---|---|---|---|---|
| TEWL (g/h/m²) | 1% | 22.63 | 16.98 | 18.22 | 14.83* |
| | 2.5% | 30.48 | 24.99 | 28.07 | 24.59 |
| Hydration (AU) | 1% | 25.99 | 38.44 | 37.32 | 45.71** |
| | 2.5% | 24.14 | 29.51 | 32.25 | 23.24 |
| Erythema (AU) | 1% | 397.73 | 352.62 | 356.13 | 321.13 |
| | 2.5% | 379.39 | 361.97 | 373.54 | 317.88 |
| Melanin (AU) | 1% | 459.92 | 446.16 | 440.75 | 292.19 |
| | 2.5% | 277.80 | 296.82 | 292.15 | 266.74 |
| Elasticity (Ua/Uf, %) | 1% | 75.18 | 77.87 | 80.06 | 75.81 |
| | 2.5% | 77.23 | 76.20 | 73.73 | 97.16 |
| Sebum (μg/cm²) | 1% | 32.20 | 14.20 | 22.70 | 21.20 |
| | 2.5% | 46.39 | 23.91 | 22.52 | 17.00 |

TEWL = trans-epidermal water loss;
Ua/Uf = relaxation/suction;
*week 0 vs week 16, p = 0.048;
**week 0 vs week 16, p = 0.024.

The 1% products led to significant increase in hydration in the combined skin conditions group, and a non-significant increase in the atopic dermatitis group. While the 2.5% led to significant increase hydration in the atopic dermatitis group, and a non-significant increase in the combined skin conditions group. In acne, the 1% products led to a significant decrease in TEWL, while the 2.5% led to a non-significant decrease. And the 1% products led to a non-significant decrease in sebum, while the 2.5% led to a significant decrease in sebum. In summary, both 1% and 2.5% products led to improvements in TEWL, hydration and sebum. However, due to the small sample sizes, it is impossible to make a conclusive comparison.

### Safety Outcomes

2 participants experienced a flare up of atopic dermatitis. Both patients were using the emulgel. One participant experienced a flare up of atopic dermatitis as the product was not hydrating enough and so had an increase in itch which led to scratching and excoriation. She stopped using the test product for a few days, then restarted adding an emollient to her daily regimen to improve hydration and her atopic dermatitis settled, with no further flare ups with continued use.

The second participant experienced an increase in itch, pain and redness in her legs 2 weeks after starting the trial. However, this coincided with a time of year when her skin typically flares. She continued using the product and the skin on her legs settled. She reported another reaction 9 weeks into the trial, where her face became puffy and red after she inadvertently touched it with product still on her hands. This worsened over several days and then slowly settled. She only reported this to us a couple weeks later, so the detailed history is unknown, and it is unclear as to whether this was a reaction to the topical product or not. She continued to use the product on her legs throughout with no further reactions.

### CONCLUSION

Treatment with topical DIM led to improved skin barrier function, increased hydration and improved disease severity in participants with atopic dermatitis and psoriasis; and improved barrier function, decreased erythema and acne severity, and improved quality of life in participants with acne. Adverse effects were minimal.

Both 1% and 2.5% products led to improvements in TEWL, hydration and sebum. However, due to the small sample sizes, it is impossible to reach definite conclusions. Both cream and emulgel led to improvements in TEWL, hydration, erythema and sebum. However, due to the small sample sizes, it is impossible to reach definite conclusions.

Even though this is an explorative study with a small sample size, the results are extremely promising. This study indicates that the use of topical DIM leads to decreased inflammation, and repair of the skin barrier with associated increased hydration, leading to improvements in acne, atopic dermatitis and psoriasis.

What is claimed is:

1. A topical composition for treating a skin disease or condition in a subject, wherein the composition comprises from about 0.1 wt % to about 10 wt % of substituted or unsubstituted diindolylmethane and a pharmaceutically acceptable carrier, and the composition provides therapeutically effective amount of the substituted or unsubstituted diindolylmethane in the subject's skin affected by the skin disease or condition.

2. The topical composition of claim 1, wherein the substituted or unsubstituted diindolylmethane is emulsified in the composition.

3. The topical composition of claim 1, wherein the composition comprises from about 0.1 wt % to about 8.0 wt % of substituted or unsubstituted diindolylmethane.

4. The topical composition of claim 1, wherein the composition comprises from about 0.1 wt % to about 5.0 wt % of substituted or unsubstituted diindolylmethane.

5. The topical composition of claim 1, wherein the composition comprises from about 0.1 wt % to about 3.0 wt % of substituted or unsubstituted diindolylmethane.

6. The topical composition of claim 1, wherein the composition comprises from about 0.5 wt % to about 2.5 wt % of substituted or unsubstituted diindolylmethane.

7. The topical composition of claim 1, wherein the composition comprises from about 0.8 wt % to about 1.2 wt % of substituted or unsubstituted diindolylmethane.

8. The topical composition of claim 1, wherein the composition comprises from about 1.0 wt % of substituted or unsubstituted diindolylmethane.

9. The topical composition of claim 1, wherein the composition comprises from about 2.0 wt % to about 3.0 wt % of substituted or unsubstituted diindolylmethane.

10. The topical composition of claim 1, wherein the composition comprises about 2.5 wt % of substituted or unsubstituted diindolylmethane.

11. The topical composition of claim 1, wherein the substituted or unsubstituted diindolylmethane is unsubstituted diindolylmethane.

12. The topical composition of claim 1, wherein the substituted or unsubstituted diindolylmethane is unsubstituted 3,3′-diindolylmethane.

13. The topical composition of claim 1, wherein the composition further comprises a retinoid compound.

14. The topical composition of claim 13, wherein the retinoid compound is a substituted or unsubstituted retinoic acid.

15. The topical composition of claim 13, wherein the retinoid compound is vitamin A.

16. The topical composition of claim 13, wherein the retinoid compound is retinyl palmitate.

17. The topical composition of claim 1, wherein the composition is essentially free of any retinoid compound.

18. The topical composition of claim 1, wherein the composition is free of any retinoid compound.

19. The topical composition of claim 1, wherein the composition is essentially free of any therapeutically active agent other than the substituted or unsubstituted diindolylmethane.

20. The topical composition of claim 1, wherein the composition is free of any therapeutically active agent other than the substituted or unsubstituted diindolylmethane.

21. The topical composition of claim 1, wherein the skin disease or condition is selected from the group consisting of atopic dermatitis, acne, and psoriasis.

22. The topical composition of claim 1, wherein the skin disease or condition is atopic dermatitis.

23. The topical composition of claim 1, wherein the skin disease or condition is acne.

24. The topical composition of claim 1, wherein the skin disease or condition is psoriasis.

25. The topical composition of claim 1, wherein the topical composition is non-irritant to skin.
